(19) United States
(12) Patent Application Publication
(10) Pub. No.: US 2019/0234902 A1
(43) Pub. Date: Aug. 1, 2019

(54) METHOD FOR DETECTING ANALYTES USING DIELECTROPHORESIS RELATED APPLICATIONS

(71) Applicant: NDSU Research Foundation, Fargo, ND (US)

(72) Inventors: Ivan T. Lima, Jr., Fargo, ND (US); Darmakeerthi Nawarantha, Fargo, ND (US); Fleming Dackson Gudagunti, Fargo, ND (US); Logeeshan Velmanickam, Fargo, ND (US)

(21) Appl. No.: 16/259,114

(22) Filed: Jan. 28, 2019

Related U.S. Application Data
(60) Provisional application No. 62/622,508, filed on Jan. 26, 2018.

(51) Int. Cl. G01N 27/447; G01N 33/543; B01L 3/00

(52) U.S. Cl. CPC G01N 27/44726; G01N 33/5438; G01N 2800/50; B01L 3/502715; G01N 2800/56; B01L 3/502761

(57) ABSTRACT

A system for determining an effect of a non-uniform electric field on a dielectric particle includes a pair of electrodes for generating the non-uniform electric field, a function generator in communication with a computer and the electrodes for changing the frequency of the electric field, and a camera in communication with a microscope and the computer for capturing a series of images of the dielectric particle in the non-uniform electric field. The computer is programmed to detect changes between the images in the series of images to determine the effect of the non-uniform electric field on the dielectric particle.

### RELATED APPLICATIONS

This application claims priority from U.S. Provisional Application Ser. No. 62/622,508 filed Jan. 26, 2018, the entire disclosure of which is incorporated herein by this reference.

### TECHNICAL FIELD

The presently-disclosed subject matter generally relates to systems and methods for sensing analytes in a sample. Embodiments of the presently-disclosed subject matter detect the effect of dielectrophoresis (DEP) force as a function of frequency on particles associated with the analyte to accurately and efficiently detect, quantify, and/or characterize analytes, including rare or small quantities of analytes, and/or to distinguish between analytes having small differences.

### INTRODUCTION

The ability to accurately and efficiently detect, quantify, distinguish, and/or characterize analytes, including rare or small quantities of analytes, has notable benefits, including benefits in research and clinical settings. For example, analysis of biological molecules, such as nucleotide and polypeptide molecules in biological samples, has significant utility for diagnosis, prognosis, and/or treatment monitoring for a number of conditions. Indeed, the early diagnosis and treatment of many conditions can significantly increase survival rates for many life-threatening conditions, such as, for example, pancreatic ductal adenocarcinoma (PDAC) (Chan et al. 2014), acute myocardial infarction (AMI) (Pawula, Altintas, and Tothill 2016), lung cancer (Xiao et al. 2012), prostate cancer (Jedinak et al. 2015), and oral cancer (Rusling et al. 2010). For example, PDAC has a 2% survival rate, which could be significantly increased if detected in early stages when it could be surgically resected (O'Brien et al. 2015).

The detection of disease biomarkers in bodily fluids, such as blood, plasma, saliva, and urine, has several advantages when compared to other methods. The analysis of the presence of disease biomarkers in bodily fluids is a non-invasive method. Meanwhile, biopsy is often a painful, expensive, and time-consuming procedure that can lead to complications (Prensner et al. 2012). Moreover, Biopsy and conventional imaging methods, including magnetic resonance and ultrasound, are not effective for the diagnosis of cancer in early stage (Altintas et al. 2011).

Therefore, there is the need for the development of a cost-effective transduction mechanism for biosensors that has the combination of sensitivity and specificity to detect the molecular biomarkers of diseases in the early stage.

Biosensors generally use biomolecules, including enzymes, proteins, nucleic acids, and antibodies as the detection agent (Pethig 2010; Pethig and Smith 2013; Qiu et al. 2015; Rusling et al. 2010; Takalkar et al. 2016; Thévenot et al. 2001; Xu et al. 2014). Biosensors can be categorized as two primary types: catalytic biosensors or affinity biosensors. The catalytic biosensors make use of active biomolecules such as enzymes that specifically react with certain target molecules or analytes (Pethig and Smith 2013). The affinity biosensors make use of the specific interaction of the analyte with the chemical receptors of another molecule (Pethig and Smith 2013). Immunosensors are affinity biosensors that make use of antibody-antigen interactions (Chen et al. 2009; Chikkaveeraiah et al. 2012; Wu et al. 2007). Antibodies are glycoprotein molecules denoted immunoglobulins that are made by plasma cells as an immune response to antigens (Pethig and Smith 2013). When used in a biosensor, the antibodies are either adsorbed or covalently bound to a surface, such as dielectric plates in the case of enzyme-linked immunosorbent essay (ELISA) or dielectric particles as in latex agglutination assay (LA) (Daniels and Pourmand 2007; Panackal et al. 2014; Velmanickam et al. 2016; Wu et al. 2007). The biological sample that may contain the target analytes is flowed, pipetted, or mixed with the immunoassay (Shekarchi et al. 1988; Velmanickam et al. 2016). Any target molecule present in the sample reacts with the antibody. In the case of the LA assay, when the target molecule reacts with the antibody, it leads to agglutination of the dielectric particles (Gella, Serra, and Gener 1991; Molina-Bolivar and Galisteo-Gonzales 2005).

There are several techniques to detect and quantify the analytes in LA and ELISA, including dielectrophoresis (DEP) and electrochemical impedance spectroscopy (Cai, Lee, and Hsing 2006; Chen et al. 2014; Gupta et al. 2012; Rodriguez, Kawde, and Wang 2005; Varshney et al. 2007; Zou et al. 2007). In DEP, various target molecules, such as antigens, antibodies, nucleotides, or cells can be detected, concentrated, isolated, or purified (Jones 2003; Pethig 2010; Pethig and Smith 2013). DEP has been used in the sorting of particles and cells. (Camacho-alanis and Ros 2015; Chu et al. 2017; Weng et al. 2016). DEP has also been used as the transduction mechanism of biosensors based on the DEP crossover frequency, in which the DEP force is equal to zero (Velmanickam, Laudenbach, and Nawarathna 2016). However, the sensitivity of that method is still not suitable for the detection of certain disease biomarkers.

A review of some prior art follows. Zhu et al., Biomicrofluidics 4, 013202 (2010) discloses an optoelectronic microdevice, which is set up to drive single microparticles and a maximum synchronous velocity MS-velocity spectrum, which is proposed for quantifying the frequency-dependent behaviors of individual neutral microparticles from 40 kHz to 10 MHz. It is one method to characterize the trapping properties of a ring trap produced optically. The optical ring is projected down to a surface that becomes optically conductive, above which the particles are dispersed in a solution. When the ring is projected in such a way that a particle is trapped, the ring is moved until reaching a speed in which the particle cannot remain trapped. What is described is a complicated and expensive setup that can enable the calculation of DEP force as a function of the frequency of the voltage source. The particles used are very large: 19 μm diameter pollen grains and 30 μm and 50 μm diameter microspheres. This method is not used as a transduction mechanism for a chemical sensor. Moreover, the method to obtain the spectrum is a very labor-intensive process, based on trial and error method.

Hughes et al., Centre for Biomedical Engineering, University of Surrey, Guildford, Surrey discloses a review of DEP theory and shows results from the use of DEP as a cell trapping/collecting mechanism. FIG. 3 of the paper shows the number of cells collected per minute as a function of the frequency. This paper highlights the dependence of the DEP trapping strength as a function of DEP, so that, for example, the frequency with the strongest trapping can be exploited in cell collection/concentration.

Adams et al, Biomicrofluidics 7, 064114 (2013) discloses the ratio between the image at the center of a quadrapole electrode (negative DEP nDEP) will concentrate the particles at the center of the electrode) with another area in the middle of the quadrapole electrode from the center as a function of the rate of change of the frequency. For any given frequency that has negative DEP (stationary measurement), this electrode concentrates the particles at the center of the electrode. This electrode can be used to calculate the average bead velocity as a function of the frequency to enable the extraction of a measurement of nDEPS, as shown in FIG. 3(d) of the paper. This method to measure negative DEP is also based on signal processing, requires 2-D image processing, and was applied to large dielectric microspheres. This paper also mentions a method to measure nDEPS to characterize blood cell particles and dielectric microspheres; however, it does not address the need to detect rare or scarce amounts of target molecules in a sample.

Modarres and Tabrizian, Sensors and Actuators B 252:391-408 (2017), disclose an extensive review of DEP with AC sources. In the studies and in the sensors reviewed, DEP was used for trapping, separation, and stretching. DEP was not used directly for sensing. Positive and negative DEP involved in these tasks, but there was no clear measurement of the negative DEP force as a function of the frequency domain (nDEPS) used as a sensing mechanism.

Mohamad et al, AIP Advances 7(1):10.1063 (2017), disclose using impedance as a transduction mechanism. This is a transduction mechanism that has much relatively low sensitivity. Impedance measurements cannot be used to detect molecules at very low concentration level.

Nakano and Ros, Electrophoresis. 34(7): 10 (2013), disclose using DEP to separate molecules using DEP traps. This method has low sensitivity and specificity. If one has a large number of molecules with two different types that undergo a different force due to DEP, this method can be used to separate out the molecules.

Emmaminejad et al, 18th International Conference on Miniaturized Systems for Chemistry and Life Sciences Oct. 26-30, 2014, San Antonio, Tex., US, disclose using negative DEP to release beads that are trapped by a bond that is weak enough to break down with negative DEP. Therefore, the purpose of negative DEP in this paper is to selectively release beads in a microfluidic system from a collection of beads. Therefore, negative DEP is used as a mechanical actuation mechanism to release the dielectric beads, not as a sensing mechanism.

Gascoyne and Vykoulal, Proc IEEE Inst Electr Electron Eng. Author manuscript; available in PMC 2009 August 13, disclose a review paper, wherein DEP was applied to selectively trap cells. It also stated that DEP lacks the necessary specificity when used directly to trap molecules.

There are a few technologies that are currently being used or are under developed for the detection of rare molecules in bodily fluids, such as nucleotide or polypeptide molecules. Sandwich Enzyme-linked immunosorbent essay (ELISA) is a commonly used method to detect target proteins. However, ELISA does not have the necessary sensitivity to detect rare analytes, such as many of the disease biomarkers in the early stage (Velmanickam et al. 2016). For example, ELISA does not have the necessary sensitivity to detect biomarkers in early-stage oral cancer. (Rusling et al. 2010).

Surface Plasmon Resonance (SPR) imaging has also been investigated in the detection of rare molecular biomarkers. (Altintas et al. 2011; Pawula et al. 2016; Puiu and Bala 2016). One limitation of SPR is the high cost that arises from the lack of reusability of the device after each assay. Essentially, a thin metal surface of the SPR system is functionalized with antibodies to the target molecules, and either needs to be functionalized periodically or treated as an expendable. This drawback limits the use of this technology in the point-of-care, especially in developing countries.

Single nucleotide polymorphism (SNP) can also serve as useful markers for diagnosis, prognosis, and/or treatment monitoring for various conditions, as well as for other purposes, such as for use connection with individualized treatment, including determination of whether particular therapeutics will be effective in a particular subject. (Maher, 2008). In a DNA sequence, a SNP represents a difference in a single nucleotide (Bush and Moore 2012). A SNP may replace a single nucleotide (A, T, G and C) with any other nucleotide (Shen, 2010). SNPs are highly stable and have only two alleles, making them useful genetic markers. Although SNP have been shown to be an useful to examine (He and Zelikovsky, 2007), LaFramboise, 2009), detecting SNPs is still difficult with existing techniques.

Direct sequencing spread spectrum is the most widely used method for the detection of SNPs because of its efficiency. (Donis-Keller, 1987). The same gene of different samples is aligned, compared with the help of PCR amplification, and analyzed for resequencing to detect SNPs. The sequencing alignment helps detect the locations of the single nucleotide and the SNPs with high accuracy. Although this technique provides high accuracy, it requires a wideband channel with a small phase distortion and large acquisition time. Another widely-used method in SNP detection is PCR. The amplification of the alleles is conducted using two pairs of primers. This process involves overlapping of the primer pairs that differentiate as between the native allele and the alternative allele for the SNP. (Kwok and Chen, 2003), Cheng, et al., IEEE trans. Nanobioscience, 2016). Another popular method for SNP detection is Restriction Fragment Length Polymorphism (RFLP). Distinctions in the DNA sequences are identified using specific restriction enzyme sites and restriction enzyme combination. (Cheng, et al., IEEE/ACM Trans. Comput. Biol. Bioinforma, 2016). This method is expensive and complex, because it needs a strong binding framework.

Mapping and Assembly with Quality (MAQ) is an application that builds maps from short reads generated by a sequencing machine. MAQ uses value-based scores to derive genotype calls according to the consensus sequence of a diploid genome. This technique is based on a Bayesian statistical model that includes error probability and mapping qualities from the quality scores of the sequence. Although MAQ is efficient and highly sensitive, there is a high probability of sequencing errors, creating reliability concerns (Talukder, et al., 2010).

The program Short Oligonucleotide Analysis Package (SOAP) is a resequencing utility, y which references between raw sequencing reads and the consensus sequence for the genome sequence to be tested. A comparison is conducted on the consensus sequence with a reference to identify SNPs. This method incorporates the data quality, alignment, and recurring experimental errors, making this method complex and with large acquisition time. (Li, eg al., 2009).

Therefore, there is the need in the art for a cost-effective systems and methods having adequate sensitivity and specificity to accurately and efficiently detect, quantify, distinguish, and/or characterize analytes, including rare or small quantities of analytes, such as molecular biomarkers of conditions in the early stage. Such markers can include, for example, nucleotide molecules, polypeptide molecules or proteins, and/or SNPs.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a system for determining an effect of a non-uniform electric field on a dielectric particle. In some embodiments, the system includes a computer, a pair of electrodes for generating the non-uniform electric field, a function generator in communication with the computer and the electrodes for changing the frequency of the electric field, and a camera in communication with a microscope and the computer for capturing a series of images of the dielectric particle in the non-uniform electric field.

In some embodiments, the computer is programmed to detect changes between the images in the series of images to determine the effect of the non-uniform electric field on the dielectric particle. In some embodiments, the computer is programmed to communicate with the function generator to induce positive dielectrophoresis (DEP). In some embodiments, the computer is programmed to communicate with the function generator to induce negative DEP.

In some embodiments, the computer is programmed to communicate with the function generator to start, stop, and step frequency; to control peak to peak voltage; to control a time interval for positive DEP frequency, and/or to control a time interval for negative DEP frequency. In some embodiments, the computer is further programmed to calculate cross over frequency. In some embodiments, the computer is further programmed to calculate the center of mass of the dielectric particle.

In some embodiments, the computer is programmed to communicate with the function generator provide a start frequency that induces positive DEP until such time as the positive DEP force results in the attraction of the dielectric particle to the electrode, at which time the computer is programmed to communicate with the function generator to change automatically to another frequency that induces negative DEP.

In some embodiments, the computer is further programmed to generate a spectrum of light intensity as a function of position for the dielectric particle. In some embodiments, the computer is further programmed to calculate velocity of the dielectric particle due to negative DEP. In some embodiments, the computer is further programmed to generate a spectrum of velocity as a function of frequency for the dielectric particle.

In some embodiments, the spectrum of velocity as a function of frequency is compared to a standard curve or a second spectrum of velocity as a function of frequency for a second dielectric particle. In some embodiments, the standard curve or the second spectrum is for a known biomarker of interest.

The presently-disclosed subject matter includes a method for measuring an effect of a non-uniform electric field on a dielectric particles. In some embodiments, the method involves (a) generating the non-uniform electric field, (b) changing frequency of the non-uniform electric field, (c) capturing a series of images of the dielectric particles during exposure to the non-uniform electric field and the changing frequency over a specified time interval, and (d) detecting changes between the images in the series of images to determine the effect of the non-uniform electric field on the dielectric particles.

In some embodiments, the method also involves providing a start frequency that induces positive DEP effect until the dielectric particle is attracted to the electrode, and then providing another frequency to induce negative DEP. In some embodiments, the method also involves using the images to calculate velocity of the dielectric particle due to negative DEP. In some embodiments, the method also involves generating a spectrum of velocity as a function of frequency for the dielectric particle.

In some embodiments, the method also involves comprising contacting the dielectric particle with a sample that possibly contains an analyte of interest, wherein the dielectric particle is functionalized with moieties for binding the analyte of interest.

In some embodiments, the method also involves comparing the spectrum of velocity as a function of frequency for the dielectric particle with a standard curve or spectrum for: (a) a dielectric particle functionalized with moieties for binding the analyte of interest that has not been exposed to a sample, and/or (b) a dielectric particle functionalized with moieties for binding the analyte of interest that has been exposed to a second sample known to contain the analyte of interest.

In some embodiments, the sample is a biological sample obtained from a subject and the analyte of interest is a biomarker for a condition of interest. In some embodiments, the results of the comparison are used for predicting, diagnosing, providing a prognosis, and/or monitoring treatment for the condition of interest.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 is a schematic diagram illustrating positive and negative DEP forces acting on particles in a non-uniform electric field.

FIG. 2 is a schematic diagram illustrating of the effect of negative DEP force on a particle in the absence and the presence of an analyte.

FIG. 3A is a picture of an exemplary experimental setup of the system described herein.

FIG. 3B is a picture of an interface with an exemplary MS Windows application for DEP spectroscopy, as described herein.

FIG. 4 is a spectroscopic curve showing the speed (pixels/sec) of the dielectric particles in the presence of DEP force as a function of frequency (kHz), wherein each pixel corresponds to 0.6 μm.

FIG. 5A-5D are a series of images captured through the application described herein, which illustrate the effect of DEP force on dielectric particles as a function of frequency. FIG. 5A is an image taken shortly after negative DEP was applied. FIGS. 5B, 2C, and 2D were taken after 80, 160, and 240 ms, respectively. The electrodes are visible in the darker regions in the picture. The bright layer visible on the edge of the electrode is formed by the accumulation of dielectric particles. As the frequency is changed to induce negative DEP the particles are repelled from the electrode. The scale bar indicates 100 μm.

FIG. 6 includes spectra of light intensity as a function of the horizontal position (distance from the edge of the electrode) in the region of interest at different time intervals (0 ms or 100 ms).

FIG. 7 includes negative DEP spectra showing a significant change in the repulsion velocity of the 0% avidin-biotin conjugated particles as compared to the 0.8% avidin-biotin conjugated particles as a function of frequency, especially in the frequency range above 1500 kHz.

FIG. 8 includes negative DEP spectra for single-stranded DNA with 16, 17, and 26 nucleotides.

FIG. 9 includes negative DEP spectra for single-stranded DNA 16 nucleotides or 17 nucleotides ending with an A, T, or G nucleotide.

FIG. 10 is a schematic diagram illustrating the sample preparation for an exemplary method of detecting an antigen, CA 19-9.

FIG. 11A-11C are a series of images captured through the application described herein, which illustrate the effect of DEP force on dielectric particles as a function of frequency. FIG. 11A is an image taken shortly after negative DEP was applied. FIGS. 11B and 11C were taken after 40 and 80 ms, respectively. The pearl shaped interdigitated electrode (PIDE) is visible as a darker region in the picture. The bright layer visible on the edge of the electrode is formed by the accumulation of the dielectric particles. As the frequency is changed to induce negative DEP the antigen bound to the dielectric particles are repelled from the electrode.

FIG. 12 is a bar graph showing velocity of the dielectric particles as a function of frequency for cutoff levels of CA19-9 for the detection of pancreatic cancer at 0 U/mL, 37 U/mL, 100 U/mL, 300 U/mL, and 1000 U/mL.

FIGS. 13A and 13B are images captured through the application described herein, which illustrate the effect of DEP force on dielectric particles as a function of frequency. FIG. 13A is an image taken shortly after negative DEP was applied. FIG. 13B depicts the repulsion of the dielectric particles away from the electrodes due to negative DEP at 40 ms.

FIG. 14 includes spectra of light intensity as a function of the horizontal position (distance from the edge of the electrode) captured shortly after the application of negative DEP and 40 ms later.

FIG. 15 includes negative DEP spectra for the single-stranded DNA molecules with the change in last nucleotide of the sequence. The error bars show the confidence interval in each individual measurement that were calculated using six measurements per frequency.

FIG. 16 includes higher resolution negative DEP spectra for the single-stranded DNA molecules with the change in last nucleotide of the sequence

FIG. 17 includes negative DEP spectra for the single-stranded DNA molecules with the change in the second-to-last nucleotide in the sequence. The error bars show the confidence interval in each individual measurement that were calculated using six measurements per frequency.

FIG. 18 includes higher resolution negative DEP spectra for the single-stranded DNA molecules with the change in the second-to-last nucleotide in the sequence.

FIG. 19A-19C includes images of exemplary electrodes at 10×, 25×, and 40× resolution, respectively. The electrodes are shown in the dark regions.

FIG. 20 includes a COMSOL Multiphysics simulation with the distribution of the gradient of the electric field intensity.

FIGS. 21A and 21B include images from an experimental demonstration of negative DEP effect through time-lapse images captured through DEP spectroscopy application. The electric field is changed from 10 kHz to 500 kHz with 10 Vp-p at t=0 ms. FIG. 21A was captured at t=0 ms and FIG. 21B was captured at t=40 ms. The interdigitated electrode is visible as a darker region in the picture. The bright layer visible on the edge of the electrode is formed by the accumulation of the sample.

FIGS. 22A and 22B include Pm distribution pattern tracing the shape of an ellipse, where b1 is the major axis and al is the minor axis. FIG. 22A was captured at t=40 ms and FIG. 22B was captured at t=80 ms.

FIG. 23 includes a negative DEP spectrum curves using new interdigitated electrodes for the change in the last nucleotide sequence for frequency varying from 500 kHz to 2000 kHz in steps of 300 kHz.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes systems and methods for sensing analytes in a sample. Embodiments of the presently-disclosed subject matter can be used to determine an effect of a non-uniform electric field on a dielectric particle, such as a dielectric particle associated with an analyte. In some embodiments, the systems and methods of the presently-disclosed subject matter can be used to accurately and efficiently detect, quantify, and/or characterize analytes, including rare or small quantities of analytes, and/or to distinguish between analytes having small differences.

Dielectrophoresis (DEP) refers to the movement of particles in a non-uniform electric field. (Pethig and Smith 2013; Varshney et al. 2007). The term “DEP force” can be used to describe the interaction between the particles and the non-uniform electric field that produces the movement.

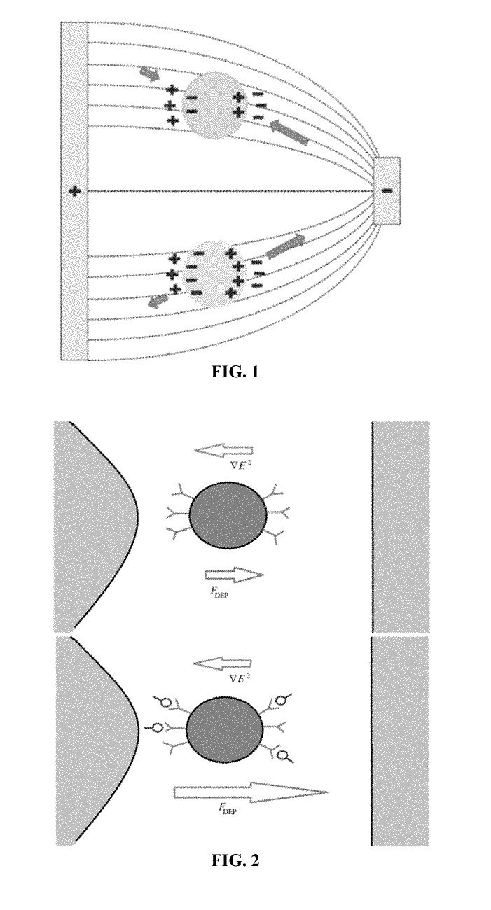

A schematic description of DEP acting on particles is shown in FIG. 1. Because of the geometry of the illustrated electrodes, the electric field magnitude near the right electrode is higher than that near the left electrode, i.e., the electric field is non-uniform. If a particle is more polarizable than the surrounding medium (the lower particle in FIG. 1), it experiences a net force towards the region with the higher electric field (the region near the right electrode in FIG. 1). This force is denoted “positive DEP.” On the other hand, if a particle is less polarizable than the surrounding medium (the upper particle in FIG. 1), it experiences a net force toward the region with lower electric field (the region near the left electrode in FIG. 1). This force is denoted “negative DEP” (Jubery, Srivastava, and Dutta 2014; Li et al. 2014; Ramos et al. 1999).

The translational forces of attraction or repulsion due to DEP arise from the interaction of the dielectric properties of particles with the non-uniform electric field. The strength and direction of the DEP force is dependent on the frequency of the electric field, the medium in which the particles are moving, the shape and size of the particles, as well as the properties of the particles. Thus, modulating frequency with DEP has been exploited in biotechnology for its capability of selectively isolating, concentrating, or purifying target particles present within a complex mixture by either attracting or repelling these target particles from the electrodes. (Lynch, Hilton, and Simpson 2006; Papagiakoumou et al. 2006; Pethig 2010). (Castellanos et al. 2003). Furthermore, the properties of the particles, and the resulting effect of DEP force, can be impacted by modifying the particle, e.g., by attaching molecules to the particle.

The time averaged DEP force on a spherical particle of radius r is given by (Pethig 2010; Pethig and Smith 2013; Velmanickam et al. 2016; Velmanickam and Nawarathna 2016):

[symbol]

F_{DEP} [symbol] =2πεₒεₘr³Re[K_{CM}(ω)]∇E²,

where ε₀ is the permittivity of the free space, εₘ is the relative permittivity of the medium, ∇E² is the gradient of the electric field intensity, and Re[K_{CM}(ω)] is the real part of the Clausius-Mossotti factor (the effective polarisability per unit volume of the particle) which is given by (Pethig 2010; Pethig and Smith 2013; Velmanickam et al. 2016; Velmanickam and Nawarathna 2016):

$${K}_{CM} ( ω ) ≡ \frac{{ɛ}_{p}^{*} - {ɛ}_{m}^{*}}{{ɛ}_{p}^{*} + 2 {ɛ}_{m}^{*}} ,$$

where εₚ* is the complex permittivity of the particle and εₘ* is the complex permittivity of the medium. The complex permittivity is related to the conductivity a and the angular frequency ω of the applied alternating current (AC) field through the following relation:

$${ɛ}^{*} ≡ ɛ - j \frac{σ}{ω} .$$

Depending on the relative values of the permittivity of the particle and the medium, the value of K_{CM}(ω) varies from −0.5 to 1. If K_{CM}(ω) is positive, the particles are attracted to regions of high electric field intensity: Positive DEP. Similarly, if K_{CM}(ω) is negative, the particles are repelled from the regions of high electric field intensity: Negative DEP. Since K_{CM}(w) is frequency dependent, both positive and negative DEP effects can be observed by changing the frequency of the applied electric field (Pethig 2010; Pethig and Smith 2013; Velmanickam et al. 2016; Velmanickam and Nawarathna 2016; Weng et al. 2016).

The crossover frequency fₓₒ is defined as the frequency at which the force changes from positive to negative DEP. It is given by (Pethig 2012; Velmanickam et al. 2016; Pethig 2010; Velmanickam and Nawarathna 2016; Weng et al. 2016):

$${f}_{xo} = \frac{1}{2 π} \sqrt{- \frac{( {σ}_{p} - {σ}_{m } ) ( {σ}_{p} + 2 {σ}_{m} )}{( {ɛ}_{p} - {ɛ}_{m} ) ( {ɛ}_{p} + 2 {ɛ}_{m} )}} ,$$

where σ_{p,m} and ε_{p,m} are the conductivity and the relative permittivity, respectively, of the particle and of the medium. The value of fₓₒ depends on the conductivity of the particle σₚ at low frequencies (<1 MHz) and on the permittivity of the particle at higher frequencies. The total conductivity σₚ of a spherical dielectric particle is given by the sum of its bulk σ_{bulk} and surface conductivity Kₛ (Pethig 2012; Velmanickam et al. 2016; Pethig 2010; Velmanickam and Nawarathna 2016).

$${σ}_{p} = {σ}_{bulk} + \frac{2 {K}_{s}}{r}$$

Since σ_{bulk} of the very small particles is negligible, the σₚ is mainly dependent on Kₛ. Depending on the size and conjugation of the dielectric particles, the value of Kₛ varies in order of magnitude and provides a means to isolate, concentrate, or separate different types of target bio particles (Camacho-Alanis and Ros 2015; Yafouz, Kadri, and Ibrahim 2014; Cui, Holmes, and Morgan 2001; Green and Morgan 1999).

The presently-disclosed subject matter includes systems and methods for determining an effect of a non-uniform electric field on a dielectric particle. As used herein, the term “dielectric particle” is used to refer to any particle or bead that can be used in a dielectrophoresis study. The presently-disclosed subject matter also includes systems and methods for detecting, quantifying, distinguishing between, and/or characterizing analytes. Such analytes can include, for example, nucleotide molecules, polypeptides, and cells. In some embodiments, the nucleotide molecule can be, for example, a DNA molecule, an RNA molecule, such as an miRNA molecule, a gene product, such as a gene product including a single nucleotide polymorphism (SNP), and/or fragments thereof. In some embodiments the polypeptide can be, for example, a protein of interest, such as an antigen. In some embodiments, the cell can be an animal or human cell. In some embodiments the cell can, for example, present an antigen of interest. As will be recognized by the skilled artisan, such analytes can be beneficial biomarkers for use in predicting, diagnosing, providing a prognosis, and/or monitoring treatment for a condition of interest. In this regard, the systems and methods described herein can be used to detect biomarkers for such use.

The terms “diagnosing” and “predicting” as used herein refer to methods by which the skilled artisan can estimate and even determine whether or not a subject has a particular condition or disease. Making a diagnosis of a condition or excluding a do not refer to the ability to predict the course or outcome of the condition with 100% accuracy. Instead, the skilled artisan will understand that the terms refer to an increased (or decreased) probability that a certain course or outcome will occur; that is, that a course or outcome is more likely to occur in a subject diagnosed with condition, or less likely to occur in a subject for which a condition is excluded. In this regard, diagnosis of a condition includes diagnosis of a high risk of a condition. In some embodiments, a high risk is a probability of at least about 50%, 60%, 70%, 80%, 90%, or greater. Excluding a condition includes a diagnosis of a low risk of a condition disease. In some embodiments, a low risk is a probability of less than about 50%, 40%, 30%, 20%, 10%, or lower.

“Making a diagnosis” or “diagnosing”, as used herein, is further inclusive of making a prognosis, which can provide for predicting a clinical outcome (with or without medical treatment), selecting an appropriate treatment (or whether treatment would be effective), or monitoring a current treatment and potentially changing the treatment. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

As used herein, the terms “treatment” or “treating” relate to any treatment of a condition of interest and include, for example, ameliorating or relieving the symptoms associated with the condition, as well as targeting underlying causes. As will be understood by those of ordinary skill in the art, when the term “prevent” or “prevention” is used in connection with a prophylactic treatment, it should not be understood as an absolute term that would preclude any sign of the condition in a subject. Rather, as used in the context of prophylactic treatment, the term “prevent” can refer to reducing the likelihood of the manifestation of the condition and/or symptoms associated therewith, such as in a subject who may be at high risk for the condition.

The presently-disclosed subject matter includes a system for determining the effect of a non-uniform electric field on a dielectric particle. The system can be used, in some embodiments, to detect, quantify, and/or characterize an analyte of interest, and/or distinguish between analytes of interest. The system can include: a pair of electrodes, a function generator, a microscope, a camera, and a computer programmed to perform unique applications. The electrodes are used for generating the non-uniform electric field, and can be any electrodes appropriate for performing DEP spectroscopy. In some embodiments, the electrodes are pearl shaped interdigitated electrodes (PIDE), as described herein. The function generator is used to change the frequency of the electric field generated by the electrodes. The microscope allows for visualization of the dielectric particles moving in the non-uniform electric field, and the camera is used to capture a series of images during this movement.

The computer is in communication with the camera and the function generator, allowing images from the camera to be collected by the computer application(s), and for the frequency of the electric field to be controlled by the computer application(s). In this manner, the computer application can detect changes between collected images in the series of images to determine the effect of the non-uniform electric field on the dielectric particle.

In embodiments of the system, various techniques can be used for visualization of particles. While various tools known to those of ordinary skill in the art can be used to visualize the particles, in some embodiments of the present invention a unique illumination set up was used. The exemplary light source includes a light mounted at a low angle, for example, an angle of about 60, 55, 50, 45, 40, 35, 30, 25, or 20°. The exemplary light source includes an LED mounted at 45° on an optical post assembly. The low angle helps prevent diffraction of the light rays from the dielectric particles, thereby reducing the amount of scattered light collected by the microscope camera. The illumination causes the light to scatter from the dielectric particles, and this scattering is captured on the microscope and by the camera resulting in the image of the dielectric particles.

The diameter of the dielectric particles and the wavelength of the LED are selected to be of the same order, making the dielectric particles appear brighter in comparison to the background. This phenomenon is due to Mie scattering that occurs when a spherical particle is of the same order as of the wavelength of the incident energy. In this regard, the diameter of the particle and the wavelength of the light source are within about 0, 20, 20, 30, 40, 50, 60, 70, 80, 90, 100, 110, 120, 130, 140, 150, 160, 170, 180, 190, 200, 210, 220, 230, 240, 250, 260, 270, 280, 290, or 300 nm. For example, in some embodiments, the light source could be about 550-575 nm while the diameter of the particles could be about 600-900 nm. In some embodiments, the light source is a green LED light. In some embodiments the light source is light having a wavelength of about 565 nm. In some embodiments the dielectric particle has a diameter of about 720-760 nm. In some embodiments, the dielectric particle has a diameter of about 740 nm.

The presently-disclosed subject matter includes a method for measuring an effect of a non-uniform electric field on a dielectric particles. The method can be used, in some embodiments, to detect, quantify, and/or characterize an analyte of interest, and/or distinguish between analytes of interest. In some embodiments, the method involves generating the non-uniform electric field, changing frequency of the non-uniform electric field, capturing a series of images of the dielectric particles during exposure to the non-uniform electric field and the changing frequency over a specified time interval; and detecting changes between the images in the series of images to determine the effect of the non-uniform electric field on the dielectric particles. In some embodiments, the system as described herein is used to practice the method.

In some embodiments, the method involves providing a frequency to induce positive dielectrophoresis (DEP), such as, for example, a frequency of about 10 kHz to about 50 kHz. In some embodiments, the method involves providing a frequency to induce negative DEP, such as, for example, a frequency of about 250 kHz to about 2 MHz.

In some embodiments, the method involves providing a start frequency that induces positive DEP until the dielectric particle is attracted to the electrode, and then providing another frequency to induce negative DEP.

In some embodiments, the method involves using the collected series of images to calculate velocity of the dielectric particle due. In some embodiments, the method involves generating a spectrum of velocity as a function of frequency for the dielectric particle.

When an experiment is conducted using the system of the presently-disclosed subject matter, the computer application can be used to communicate with the function generator. For example, the application can be used to start, stop, and step frequency; to control peak to peak voltage; to induce and/or control a time interval for positive DEP frequency, and/or to induce and/or control a time interval for negative DEP frequency. The function generator can provide a frequency of about 10 kHz to about 50 kHz to induce positive DEP. The function generator can provide a frequency of about 250 kHz to about 2 MHz to induce negative DEP.

For certain experiments, for example, application communicates with the function generator provide a predetermined start frequency that induces positive DEP, which can be maintained for a specified time interval or until such time as the positive DEP force results in the attraction of the particles to the electrode. Once the particles are accumulated at the electrode, the application can automatically change to another frequency that induces negative DEP. The negative DEP can be maintained for a specified time interval or until such time as a predetermined stop frequency is reached.

Although the system can be used to capture images during both positive and negative DEP, in some embodiments the computer application can be in communication with the camera to capture a series of images while negative DEP is being applied until the stop frequency is reached, e.g., 250 kHz-3200 kHz, 500 kHz-2000 kHz, etc. as desired. The application can be used to automatically control frequency step, e.g., a frequency step of 300 kHz from 500 to 800 kHz, and from 800 to 1100 kHz, etc. The application can also be used to automatically control the time intervals for image collection, e.g., t=0, t=40 ms, t=80 ms, etc. The resulting images provide information about particles being repelled from the electrode by negative DEP.

The computer application can be used to process the collected images from an experiment. In this regard, the light intensity of the particles in a series of at least two images can be compared and the difference in position of the particles can be used to determine velocity of the particles as they are repelled (or attracted) to an electrode. In some embodiments, the computer application is used to generate a spectrum of light intensity as a function of position for the dielectric particle. In some embodiments, the spectrum of light intensity as a function of position is for a first time point and the spectrum is compared to a second spectrum of light intensity as a function of position for a second time point. In some embodiments, the computer application is used to generate a spectrum at particular time points following application of negative DEP, e.g., t=0, t=40 ms, t=80 ms, etc.

The computer application can be used to calculate velocity of a dielectric particle in the non-uniform electric field, such as velocity due to negative DEP. The velocity can be calculated by comparing the position of the particle at a first time point and to the position of the particle at a subsequent time point(s). In some embodiments, the computer application is used to generate a spectrum of velocity as a function of frequency for the dielectric particle. For example, in some embodiments, the spectrum shows velocity of the particle as a function of frequency over a range of frequencies, e.g., 250 kHz-3200 kHz, 500 kHz-2000 kHz, etc. as desired. The computer application can be used to control the frequency step over the desired range, e.g., a frequency step of 200 kHz from 500 to 700 kHz, from 700 to 900 kHz, from 900 to 1100 kHz, from 1100-1300 kHz, from 1300-1500 kHz, etc.

The computer can also be used to calculate cross over frequency and/or the center of mass of the dielectric particle.

The computer application can also be used to compare a spectrum for a first dielectric particle to another spectrum, such as a standard curve from a database or a generated spectrum of a second dielectric particle. In this regard, the an analyte associated with the dielectric particle can be detected, quantified, characterize, and/or distinguished from other analytes. For example, reference is made to FIG. 2, which includes a schematic representation of the change in the DEP force due to the binding of target molecules to antibodies at the surface of a dielectric particle. The upper panel illustrates a dielectric particle that has been functionalized with an antibody, which has not been placed in contact with a sample that could contain an antigen for the antibody. The antigen free dielectric particle is placed in a non-uniform electric field. A spectrum of velocity as a function of frequency can be obtained using the system as disclosed herein. The spectrum could be a standard curve that has been stored in a file or database for reference when an experiment is conducted using the functionalized dielectric particle for detecting the antigen, or it could be a spectrum that is obtained at such time as the experiment for detecting the antigen is conducted.

In any event, the functionalized dielectric particle can be placed in contact with a sample that might contain the antigen before obtaining a spectrum of velocity as a function of frequency. If there is no antigen in the sample, the spectrum after exposure to the sample will be the same as the standard spectrum/spectrum obtained before exposure to the sample. However, as illustrated in the bottom panel of FIG. 2, if the antigen is in the sample, it will bind the antibody and impact DEP force when the particle is placed in the non-uniform electric field, resulting in a spectrum that is distinct from the standard spectrum/spectrum obtained before exposure to the sample. As will be appreciated by the skilled artisan, the sample can be a biological sample, such as urine, blood, or plasma.

As will also be appreciated by one of ordinary skill in the art, in a similar manner, various spectra can be obtained and compared to detect, quantify, characterize, and/or distinguish analytes. For example, in some embodiments, an analyte of interest if attached to the dielectric particle. In some embodiments, a moiety for binding an analyte of interest is attached to the dielectric particle. Such attachment can be covalent or non-covalent (e.g., forming a complex). For another example, some embodiments make use of a DEP spectrum or standard/calibration curve for a particular analyte of interest.

In some embodiments, the analyte of interest is a biomarker for use in predicting, diagnosing, providing a prognosis, and/or monitoring treatment for a condition of interest. As will be appreciated by the skilled artisan, the biomarker could be, for example, a cell, a polypeptide molecule, a nucleotide molecule, such as a DNA molecule or an RNA molecule, e.g., miRNA molecule, or a nucleotide molecule including a single nucleotide polymorphism (SNP).

In this regard, the method and/or system disclosed herein can be used to detect the biomarker in a biological sample obtained from a subject. In some embodiments, it can be useful to compare a spectrum obtained after contacting a functionalized dielectric particle with the sample from the subject with a standard curve for the biomarker.

For example, in some embodiments, it can be useful to have a DEP spectrum, such as a negative DEP spectrum, for known biomarkers of conditions. In some embodiments, such DEP spectra can be provided as a function of frequency and/or concentration of the biomarker. Examples of such biomarkers include, but are not limited to: troponin T, troponin I, myoglobin, and KCMB (cardiovascular diseases); CA 19-9, lamininyC, and CA 125 (pancreatic ductal adenocarcinoma (PDAC)); PSA, β-2 microglobulin, pepsinogen 3 group 1, and intestinal mucin (lrostate cancer); CEA, hEGR and CA 15-3 (lung cancer); and selected of MicroRNA biomarkers for tumor classification (Rosenfeld et al. 2008).

In some embodiments, depending on the results obtained from the method and/or system, treatment for the condition of interest can be administered to the subject.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

The presently-disclosed subject matter is further illustrated by the following specific but non-limiting examples. The following examples may include compilations of data that are representative of data gathered at various times during the course of development and experimentation related to the present invention.

### Examples

### Example 1: Dielectrophoretic (DEP) Spectroscopy Application and System

DEP cross-over frequency has been used in detecting and quantifying biomolecules. A manual procedure is commonly used to estimate the cross-over frequency of biomolecules. Therefore, the accuracy of this detection method is significantly limited. To address this issue, the present inventors designed and tested an automated procedure to carry out DEP spectroscopy An exemplary embodiments of the method is described and tested in this example. The method efficiently measures the effect of the DEP force through a live video feed from the microscope camera and performs real-time image processing to efficiently measure the effect of DEP force on dielectric particles. This allows for enhanced accuracy in determining DEP crossover frequency and DEP spectroscopic curves for dielectric particles in a conductive solution or medium, which has application for detecting, quantifying, and characterizing biomolecules attached to the dielectric particles.

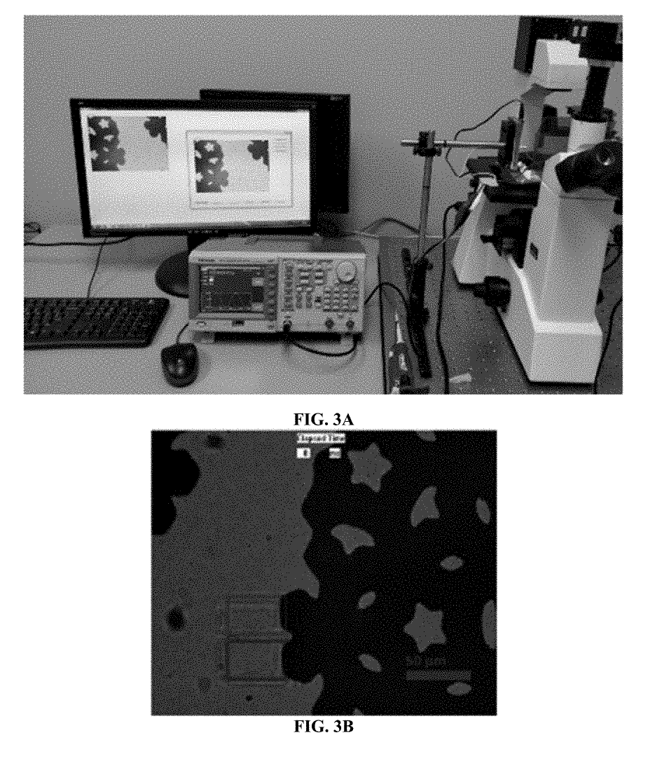

The exemplary system disclosed and tested in this example uses a Microsoft Foundation Classes (MFC) application in Visual C++ for a Microsoft Windows operating system. The software/application interacts with and controls a USB video class (UVC) standard compliant microscope camera, Tektronix AM series function generators, and a pearl shaped interdigitated electrode. This application captures and displays time lapse images from the sequence of video frames recorded at 25 frames per second by the UVC standard compliant microscope camera. With the application controlling the function generator, the frequency is swept at low and high values to apply positive and negative DEP. The experimental set up is as shown in FIG. 3A.

Because the interest is in efficiently analyzing the effect of the DEP force on dielectric particles in a sample, regions of interest are identified in the video where strong DEP effect is visible. Those regions of interest are marked, e.g., with rectangles, squares, or parallelograms, as shown in FIG. 3B. The image area within these regions of interest is processed and recorded for efficiently characterizing DEP force and calculating DEP spectroscopic curves.

The microscope camera extracts pixel and color information from the regions of interest of the live video using a real-time image processing algorithm. With these data the drift velocity due to DEP force as a function of frequency of the electric field is calculated using the algorithm. The time lapse images captured for the drift velocity calculation can be set to desired time intervals. The least possible capture time that can be set for the exemplary embodiment of this example is 40 ms, is due to the processing speed of the hardware and the frame rate at which the video is captured.

Different time intervals can also be specified for positive and negative DEP analysis. Since the suspended particles take some time to accumulate along the edges of the electrode when positive DEP is observed, additional time is needed to analyze this effect. However, in case of negative DEP, the accumulated particles along the edge of the electrode are readily repulsed as the frequency is changed. Thus, negative DEP requires much less time for examination.

Pearl-Shaped Interdigitated Electrode.

Although the direction of movement for the dielectric particle depends upon the relative polarizability of the dielectric particle and the medium, a set of electrodes is necessary for creating a region in which the particle experiences DEP force. (Gudagunti, et al. Chemosensors, 2018). The electrode creates a near field profile to create dielectric particle movement under the application of electric field. In the exemplary, system described in this example, pearl shaped interdigitated electrodes designed using AutoCAD and the generated electric field gradient was validated using COMSOL Multiphysics. The electrode was fabricated on a commercially available glass wafer with standard fabrication procedures involving photolithography, metal sputtering and lift-off procedures using 1000 Å thick gold film in the microfabrication facilities at North Dakota State University. Using COMSOL the maximum electric field of the electrode was determined to be 1.8×10⁴ V/m, with gradients as high as 3×10¹² V²/m². No variation on the root mean square of the electric field was observed, and the electric field gradient produced by this electrode with respect to frequency ranged from several kHz to few Mhz. A constant electric field is applied on the particle and the medium in all frequency ranges, thus making the DEP depend only on the real part of the Clausius-Mossotti factor. (Kirmani, et al., J. Biomed. Opt., 2017).

Side Illumination Set Up.

A custom-made illumination set up was built using a 10 mm green LED light mounted at 45° on optical post assemblies. The set up was designed based on the principle of dark field microscopy, in which the object is brightly illuminated against a dark background. The low angle helps prevent diffraction of the light rays from the dielectric particles thus reduces the amount of scattered light collected by the microscope camera. The sample is pipetted on the surface of the electrode, the illumination makes the light scatter from the biotin bound dielectric particles and this scattering is captured on the microscope camera resulting in the image of the biotin based PM. The diameter of the biotin functionalized dielectric particles are of the same order as the wavelength of the green LED making the biotin bound dielectric particles appear brighter in comparison to its background. This phenomenon is due to Mie scattering where in the sample spherical particle is of the same order as of the wavelength of the incident energy.

Frequency Sweep and Real-Time Image Processing.

The drift velocity is measured from the point of application of positive DEP to the negative DEP at the applied frequency. The positive DEP is applied to attract the dielectric particles on the edge of the electrode at lower frequency and by the application of the higher frequency thus generating the negative DEP the dielectric particles are repelled from the electrode edge. The custom-made software is built in such a way it sweeps a set of alternating frequency generating positive and negative DEP. The experiments can be run at a minimum frequency of 500 kHz and a maximum of 2 MHz in linear steps. The peak to peak voltage can be set to 10 V.

A preliminary set of experiments can be conducted to determine the choice of time intervals for the application of negative and positive DEP. The time interval set for the positive DEP can be, e.g., 1000 ms, and for the negative DEP it can be, e.g., 40 ms, per frequency measurement. With the application of positive DEP for 1000 ms at lower frequency of 10 Khz the dielectric particles accumulates on the edge of the interdigitated electrodes. The software automatically switches the frequency to a higher value after 1000 ms, inducing negative DEP effect for the set 40 ms. The cycle repeats with the application of positive DEP and negative DEP along with the image acquisition and its pixel value until the stop frequency is reached.

The function generator used in the experiments are Tektronix AFG series and this is connected to the to the computer via USB port. The software design lets us set the time intervals for the application of both positive and negative DEP effect and lets us choose the sequence of frequency to be applied generating the DEP effect. To improve the computational speed, the image acquisition data is saved into the temp file and it is dumped into the hardware once the stop frequency is reached. An Interchangeable Virtual Instruments Foundation (IVI) compliant device driver from LabWindows™/CVI environment was modified to Microsoft Visual C++. This modified device driver, along with TekVISA (Virtual Instrument Software Architecture) connectivity software, was used to access and control the function generator from Microsoft Visual C++ program.

The spectrum measurement can be based on the drift velocity due to negative DEP as a function of the frequency. This is because the negative DEP provides a broader frequency range for the dielectric particles used in the experiments. The automation gets simplified as it is easier to identify the starting point of the dielectric particles before measuring the drift velocity.

### Example 2: Rare Analyte Quantification Through DEP Spectroscopy

The variation of DEP force on biotin-avidin conjugated dielectric particles at various frequencies was studied using the system described in Example 1. Biotin functionalized dielectric particles with 0.74 μm diameter (10,000 biotin molecules on each bead surface) were purchased from Spherotech Inc. These biotinylated beads were conjugated with fluorescently labelled avidin molecules (1 mg/mL) purchased from Vector Labs Inc. using the procedure recommended by the manufacturer. In order to achieve 0.8% avidin-biotin conjugation (i.e., 80 avidin molecules attached to 10,000 biotin molecules on the surface of dielectric particles), 24 nL of avidin solution and 10 μl biotin functionalized dielectric particles were incubated for 30 min at room temperature. The solution was centrifuged at 5000 rpm for 12 min to separate functionalized beads from the solution. After centrifugation, the supernatant was removed and 390 μl of 0.01× diluted phosphate buffer saline (PBS) with conductivity 0.01 S/m was added. The 10 μl of the prepared conjugated bead solution was pipetted onto the PIDE electrode for microscopic observation during the experiment. The 0% solution contained only biotin functionalized dielectric particles.

For the experiment a clean PIDE electrode was mounted on a OMFL600 low power microscope and a side illumination technique as employed for observations. A custom-made green LED lamp illuminating from an angle of incidence of 45 degrees was used. This angle of incidence reduces the amount of light collected by the camera that does not result from Rayleigh scattering from the beads. When 10 μl of the prepared beads solution was pipetted onto the electrode, the scattered light from the beads illuminated the electrode and was refracted towards the microscope objective, resulting in a sharp image of the beads without the use of fluorescence. Since the diameter of the beads (˜740 nm) was of the same order of magnitude as the wavelength of the green LED (565 nm), the beads appear very bright on a dark background due to Rayleigh scattering. The PIDE electrode was then connected to the Tektronix AFG 3021B function generator.

The DEP spectroscopy application software was used for observation and recording of the results during the experiment. The DEP spectroscopy measurement was initiated. At the beginning, the function generator was automatically set to a positive frequency of 10 kHz to establish positive DEP. Low frequency electric fields (<50 kHz) induce positive DEP, whereas high frequency electric fields (>250 kHz) induce negative DEP force for the dielectric particles suspended in the PBS buffer. (Velmanickam, et al., Phys. Rev. 2016). The positive DEP force resulted in the attraction of the beads towards the edge of the electrodes. A clear line of bright white beads can be seen forming at the edge of the electrodes. Once the beads form a layer at the edge of the electrode, the application automatically switched the frequency to a preset negative value in order to observe the negative DEP spectroscopy. The negative DEP force resulted in the quick repulsion of the beads from the edge of the electrode. The repulsion of the beads was tracked and recorded through the application and the speed (pixels/second) of the repulsed beads as they traveled away from the electrode edge was calculated through image processing.

A relationship between the speed of repulsion of the beads and the applied frequency of the electric field was observed. Upon switching to higher frequency electric fields from the application, the negative DEP force was increased, resulting in a higher speed of repulsion from the edge of the electrode. While switching the frequency, all other experiment parameters and conditions were maintained constant. The obtained DEP spectroscopy results are shown in FIG. 4. A significant change in the repulsion speed of the beads between 0% avidin-biotin conjugation (i.e., only biotin functionalized dielectric particles) and 0.8% conjugation (80 avidin molecules attached per biotin functionalized dielectric particle) was observed, especially in the frequency range above 1500 kHz. This means that the experimental setup can detect as little as 80 avidin molecules attached on the biotin functionalized dielectric particle surface.

The dielectric particles also experience hydrodynamic drag force due to friction, which is opposite in direction to the DEP force. Since the hydrodynamic drag force also depends on the size of the particle, biotin functionalized dielectric particles with higher concentrations of attached avidin experienced higher drag force. The observed increase in speed for these beads means that the increase in DEP force was greater than the increase in the hydrodynamic drag force. The graph in FIG. 4 can be considered as a calibration curve for sensing and identification of avidin in biological samples in a medium with the same conductivity.

The method illustrated in this example can help in the detection and quantification of rare analytes in biological samples. DEP crossover frequency depends on the electrokinetic properties of the analytes and the biological sample. DEP crossover frequency has been utilized in detection and quantification of biomolecules like proteins, nucleic acids etc. (Velmanickam, et al., Phys. Rev. 2016). Currently, manual procedures are often employed to estimate the crossover frequency of biomolecules severely limiting their accuracy and application in point of care diagnostic devices. A few automated techniques available for quantification of DEP force and estimation of crossover frequency are too complex, time consuming and require costly equipment. The method disclosed herein is efficient in terms of cost and duration of the experiment and has the ability to produce reliable results with a small volume of the sample.

### Example 3: Rare Analyte Quantification Through DEP Spectroscopy

The system of Example 1 was used for further studies. In this example, the application was used to convert captured frames into greyscale and to perform real-time image processing to obtain useful information. For this example, the start frequency was 500 kHz, the frequency step was 300 kHz, and the stop frequency was 2 MHz, which produces a negative DEP spectrum with six measurements. The peak-to-peak voltage value was 10 V. The time interval for positive DEP was 30 seconds and the time interval for negative DEP was 4 seconds per frequency measurement.

To measure the negative DEP spectrum, the experiment starts with a certain frequency fₚ that induces strong positive DEP effect for a specified time interval to concentrate the dielectric particles at the edges of the electrodes. When the time interval of the positive DEP elapses, the frequency of the function generator is automatically changed to the first frequency f_{n,1} that induces negative DEP effect for the specified time interval. As the time interval for frequency f_{n,1} is elapsed, the frequency is switched back to the frequency fₚ, to transport the beads to the region with high electric field at the edges of the electrodes. Then, the next negative DEP frequency f_{n,2} is applied, which corresponds to the previous negative DEP frequency f_{n,1} incremented by the frequency step size. This cycle goes on until the frequency f_{n,N} reaches or exceeds the stop frequency. All frequency switching by the function generator is done automatically by the application.

Experiments were performed with a PID electrode array that was designed and drawn to scale in AutoCAD, validated in the COMSOL Multiphysics software package and fabricated on commercially available glass wafer using photolithography, metal sputtering and lift-off procedures using 1000 Å thick gold film. The electrode is capable of generating the maximum electric field of 1.8×10⁴ V/m, which is sufficient to polarize the dielectric particles for microscopic observation of DEP. Average electric field and electric field gradients do not vary with frequency in the electrode. (Velmanickam, et al., Phys. Rev. 2016). This ensured that the dielectric particles are subjected to the same electric field gradients in all frequencies and their DEP behavior is only dependent on the value of Re[K_{CM}(ω)], which is significantly dependent on the number of analyte molecules attached to the bead surface.

The variation of DEP force on biotin functionalized dielectric particles, with and without a small number of avidin molecules per bead, was studied at various frequencies. Biotin-functionalized dielectric particles with 0.74 μm diameter (10,000 biotin molecules on each bead surface) were purchased from Spherotech Inc. These biotinylated beads were conjugated with fluorescently-labelled avidin molecules (1 mg/ml) purchased from Vector Labs Inc. through manufacturer suggested procedure.

To achieve 100% avidin-biotin conjugation (i.e. 10,000 biotin molecules on the surface of dielectric particles attached to 10,000 avidin molecules) 3 μL of avidin solution and 10 μL biotin functionalized dielectric particles were incubated for 30 min at room temperature. The solution was centrifuged at 5000 rpm for 12 min to separate functionalized beads from the solution. After centrifugation the supernatant was removed and 390 μL of 0.01× diluted phosphate buffer saline (PBS) with conductivity 0.01 S/m was added. The prepared conjugated bead solution (10 μL) was pipetted onto the commercially available glass slide for microscopic observation during the experiment. Similarly, 0.8% avidin-biotin conjugated solutions were prepared by diluting the avidin solution appropriately and keeping other parameters (incubation time, temperature, and centrifuge velocity) constant. The 0% solution contained only biotin functionalized dielectric particles. Experiments were conducted for 0% and 0.8% avidin-biotin conjugated solutions.

For the experiment a clean PID electrode array was mounted on a OMFL600 low power microscope and a novel side illumination technique was used for observations. A custom-made green LED lamp illuminating from an angle of incidence of 45 degrees was used. This angle of incidence reduces the amount of light collected by the camera that does not result from Rayleigh scattering from the beads.

When 10 μL of the prepared beads solution was pipetted onto the electrode, the scattered light from the beads illuminated the electrode and was refracted towards the microscope objective, resulting in a sharp image of the beads without the use of fluorescence. Since the diameter of the beads (˜740 nm) is of the same order as the wavelength of the green LED (565 nm), the beads appear very bright on a dark background due to Rayleigh scattering. The use of an LED light source with a 45° incidence angle, which was simple and cost-effective, precluded the need of a complicated fluorescent enabled sample and fluorescent microscope for the experiments. The PID electrode array was then connected to the Tektronix AFG 3021B function generator.

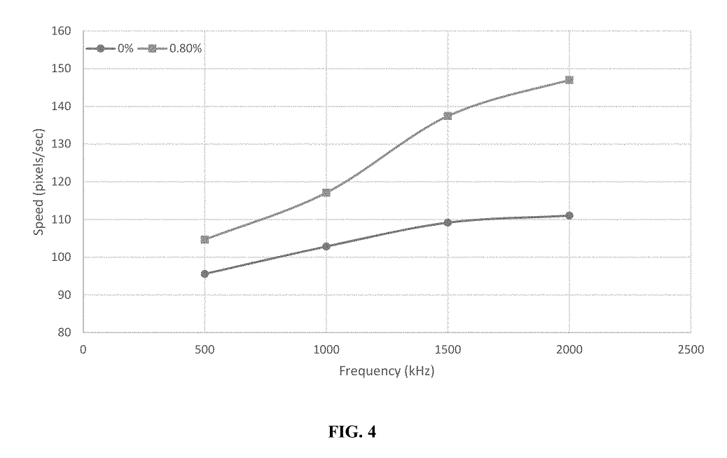

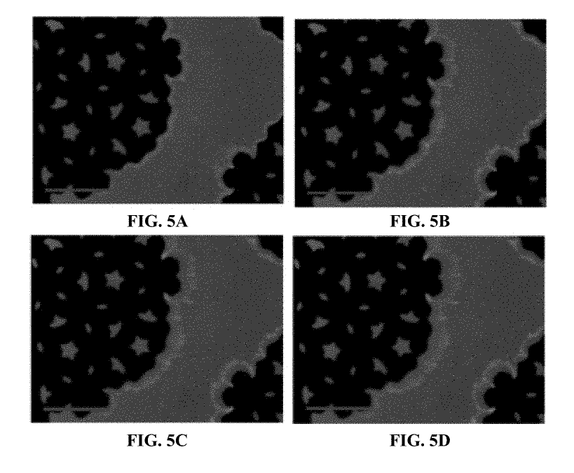

The application was used for observing and recording of the results during the experiment. At the beginning, the function generator was automatically set to a positive frequency of 10 kHz to cause positive DEP. Low frequency electric fields (<50 kHz) induce positive DEP whereas high frequency electric fields (>250 kHz) induce negative DEP force for the dielectric particles suspended in the PBS buffer. (Velmanickam, et al., Phys. Rev. 2016). The positive DEP force resulted in the attraction of the beads towards the edge of the electrodes. A clear line of bright white beads can be seen forming at the edge of the electrodes in FIG. 5A, which includes an image taken shortly after negative DEP was applied. FIGS. 5B, 5C, and 5D were taken after 80, 160, and 240 ms, respectively. Once the beads form a layer at the edge of the electrode, the application automatically switched the frequency to one of the frequencies that produce negative DEP force. The negative DEP force produces a strong repulsion force on the beads from the edges of the electrodes. The repulsion velocity of the beads was tracked and recorded through the application and the velocity (μm/second) of the repulsed beads as they traveled away from the electrode edge was calculated through image processing.

The variation of the light intensity was measured with respect to the horizontal position in the region of interest at different time intervals as shown in FIG. 6. The peak light intensity observed in the image corresponds to the position of the functionalized beads. The velocity of repulsion is calculated by processing two images, one captured at positive DEP and the other just after switching to negative DEP. The shift in the center of mass of the light intensity, which is proportional to the local concentration of the dielectric particles, is calculated for both images and is used to calculate the velocity of the repulsion due to negative DEP. The results for ten repetitions of the experiment are recorded in Tables 1 and 2. These ten experiments were conducted with four different 10 μL droplets for each of the two cases considered: 0% and 0.8% avidin-biotin conjugation. The PID electrode array was washed with deionized water before a new droplet was applied.

TABLE 1
Experiment results with 0% avidin-biotin binding.

| | Frequency (kHz) | | | | | |
|---|---|---|---|---|---|---|
| | 500 | 800 | 1100 | 1400 | 1700 | 2000 |
| | Velocity | | | | | |
| Experiment | (μm/s) | | | | | |
| 1 | 58.78 | 63.52 | 63.86 | 66.59 | 67.42 | 68.79 |
| 2 | 57.66 | 62.68 | 64.12 | 66.05 | 67.01 | 67.97 |
| 3 | 58.35 | 63.49 | 63.64 | 66.19 | 67.79 | 68.96 |
| 4 | 57.08 | 63.46 | 64.10 | 66.32 | 67.37 | 68.88 |
| 5 | 57.55 | 63.43 | 63.70 | 66.19 | 67.57 | 68.74 |
| 6 | 57.47 | 63.55 | 63.68 | 66.17 | 67.63 | 68.78 |
| 7 | 57.00 | 63.35 | 63.92 | 66.48 | 67.51 | 68.89 |
| 8 | 57.53 | 63.08 | 63.71 | 66.04 | 67.62 | 68.94 |
| 9 | 57.23 | 63.12 | 64.06 | 66.50 | 67.39 | 68.72 |
| 10 | 57.27 | 63.38 | 63.97 | 66.59 | 67.42 | 68.82 |
| Average | 57.59 | 63.30 | 63.87 | 66.31 | 67.47 | 68.75 |
| Std. Dev. | 0.56 | 0.27 | 0.19 | 0.21 | 0.21 | 0.29 |
| Rel. Error | 0.98 | 0.43 | 0.29 | 0.32 | 0.31 | 0.42 |
| (%) | | | | | | |

TABLE 2
Experiment results with 0.8% avidin-biotin binding.

| | Frequency (kHz) | | | | | |
|---|---|---|---|---|---|---|
| | 500 | 800 | 1100 | 1400 | 1700 | 2000 |
| | | | Velocity | | | |
| Experiment | | | (μm/s) | | | |
| 1 | 62.70 | 65.68 | 72.02 | 80.86 | 84.53 | 88.42 |
| 2 | 62.93 | 65.70 | 72.16 | 80.72 | 84.29 | 88.40 |
| 3 | 62.57 | 65.48 | 72.53 | 80.75 | 84.35 | 88.39 |
| 4 | 62.57 | 65.60 | 72.33 | 80.99 | 84.41 | 88.36 |
| 5 | 62.87 | 65.82 | 72.26 | 80.78 | 84.08 | 88.52 |
| 6 | 62.75 | 65.68 | 72.17 | 80.45 | 84.39 | 88.57 |
| 7 | 62.81 | 65.63 | 72.06 | 80.56 | 84.53 | 88.68 |
| 8 | 62.50 | 65.68 | 72.53 | 80.55 | 84.52 | 88.48 |
| 9 | 62.69 | 65.87 | 72.44 | 80.59 | 84.23 | 88.43 |
| 10 | 62.63 | 65.71 | 72.34 | 80.97 | 84.56 | 88.36 |
| Average | 62.70 | 65.69 | 72.29 | 80.72 | 84.39 | 88.46 |
| Std. Dev. | 0.14 | 0.11 | 0.18 | 0.18 | 0.15 | 0.10 |
| Rel. Error | 0.22 | 0.17 | 0.25 | 0.23 | 0.18 | 0.12 |
| (%) | | | | | | |

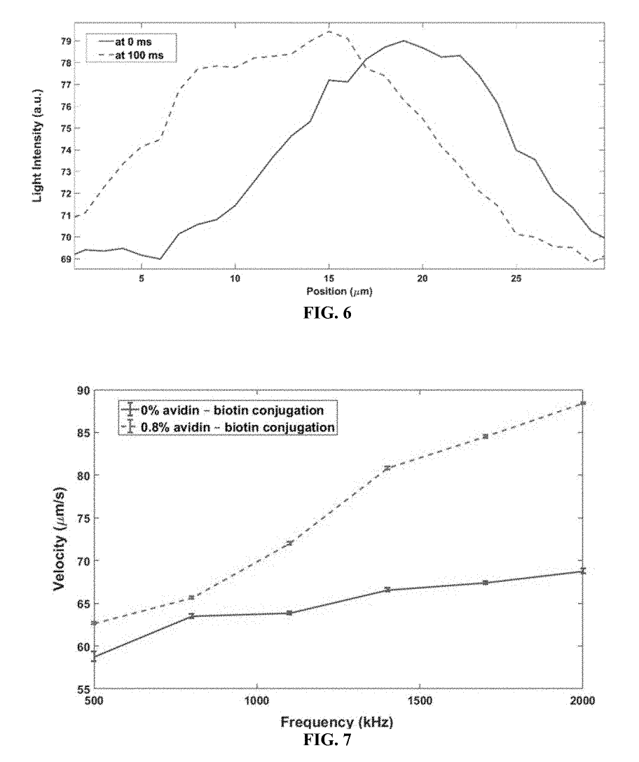

A relationship between the velocity of repulsion of the beads and the applied frequency of the electric field was observed. Upon switching to higher frequency electric fields, the negative DEP force was increased, resulting in a higher velocity of repulsion from the edge of the electrode. While switching the frequency, all other experiment parameters and conditions were maintained constant. The negative DEP spectrum obtained from the results shown in Tables 1 and 2 is shown in FIG. 7, along with the standard deviation of a measurement. A significant change in the repulsion velocity of the beads between 0% avidin-biotin conjugation (i.e. only biotin functionalized dielectric particles) and 0.8% conjugation (an average of 80 avidin molecules attached per biotin functionalized dielectric particle) was observed, especially in the frequency range above 1500 kHz. This means that the experimental setup can detect as little as 80 avidin molecules attached on the biotin functionalized dielectric particle surface.

The dielectric particles also experience hydrodynamic drag force due to friction, which is opposite in direction to the DEP force. Since the hydrodynamic drag force also depends on the size of the particle, biotin-functionalized dielectric particles with higher concentrations of attached avidin experienced higher drag force. The observed increase in velocity for the beads with 0.8% of avidin-biotin conjugation, when compared to the beads without avidin, means that the increase in DEP force was greater than the increase in the hydrodynamic drag force due to the binding of avidin. The graph in FIG. 7 can be considered as a calibration curve for sensing and identification of avidin in biological samples in a medium with the same conductivity.

The method illustrated in this example provides an automated technique for the detection and quantification of rare analytes in biological samples based on negative DEP spectroscopy. An improvement by one order of magnitude was achieved in the detection limit of avidin. The image acquisition system collects the Rayleigh scattering from dielectric particles illuminated by an LED source with 45° incidence. Therefore, there is no need to use fluorescence markers and fluorescence filters in this method.

### Example 4: DNA Identification Using DEP Spectroscopy

The system of Example 1 was used for further studies. In this example, the application was used to identify DNA molecules. The method was demonstrated using DNA molecules with 16, 17, and 26 base pairs of nucleotides.

The software application was used to retrieve pixel information of captured video frames at areas of interest to characterize DEP force. The best possible frequency range was calculated through a series of experiments, the start frequency was set to 500 kHz, and the stop frequency was set to 3200 kHz. The applied voltage was set to 10 V peak to peak. 10 μL of sample solution were pipetted on to the microelectrodes. SS DNA molecules were conjugated to commercially available biotin functionalized dielectric beads with a 740 μm diameter. The side illumination set up at 45° produced a good contrast for the imaging of the dielectric beads due to scattering. Since the diameter of the dielectric beads attached with the SS DNA sample is of the same order as the wavelength of the green LED, the beads appear very bright on a dark background due to light scattering. The imaging method based on light scattering from the dielectric beads precludes the need of an intricate fluorescent labels and fluorescent microscope for the experiment, thereby simplifying the experimental setup.

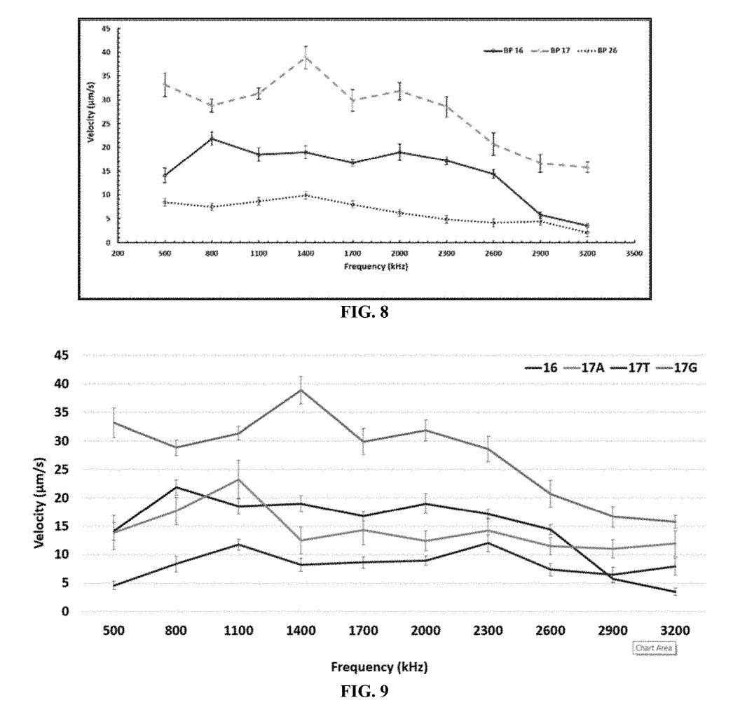

The light intensity as a function of the distance from the edge of the electrode from the microscope images was used to calculate the velocity of repulsion due to the application of negative DEP force to the beads for SS DNA samples with 16, 17, and 26 base pairs, which were used to test the method. A significant difference in the negative DEP force was observed for the SS DNA samples investigated with respect to frequency of the electric field produced by the pearl shaped interdigitated gold microelectrodes. FIG. 8 shows the negative DEP spectrum curves of SS DNA with 16, 17 and 26 base pairs respectively. FIG. 9 shows the negative DEP Spectrum curves of SS DNA of single-stranded DNA with 16 base pairs and 17 base pairs ending with an A, T, or G nucleotide.

The method illustrated in this example provides an automated technique illustrating the dependency of negative DEP spectrum on the length of the SS DNA samples and the nucleotides in the SS DNA samples. The difference in the spectrum of the velocity of repulsion, which is proportional to the DEP force, was calculated for SS DNA with 16, 17 and 26 base pairs, and SS DNA with 16 and 17 base pairs ending with an A, T, or G nucleotide, which were conjugated with biotin functionalized dielectric beads for a frequency range of 500 kHz to 3200 kHz. A significant change in the repulsion velocity of the beads was detected, illustrating that the method can be used to differentiate single nucleotide differences.

### Example 5: Protein Detection Using DEP Spectroscopy

The system of Example 1 was used for further studies. In this example, the application was used to detect a protein. In particular, the protein identified in this example was carbohydrate antigen CA 19-9, a the biomarker that can be used in the detection of pancreatic cancer (Del Villano et al., 1983). CA 19-9 is currently the only biomarker that has been approved by the U.S. Food and Drug Administration (FDA) for accurate diagnosis and monitoring of pancreatic cancer (Duffy et al., 2009). A detailed literature review of pancreatic cancer was conducted by (Poruk et al., 2013) and they demonstrated various cutoff levels of CA 19-9 used as a screening tool in asymptomatic patients for the Diagnosis of Pancreatic Cancer. Many techniques have been proposed to detect CA 19-9. Most of the investigative procedures involve CA 19-9 monoclonal antibody 1116-NS-19-9 in recognizing CA 19-9 as an explicit probe (Passerini et al., 2012). A solid-phase radio immunometric sandwich assay that can readily react with a carbohydrate antigenic determinant CA 19-9 at low concentrations has been used. This has led to enzyme-linked immunosorbent assay based techniques using electrochemical assays, photo-electrochemical assays and fluorescent based assays (Zhu et al., 2016). Recent advances have detected CA 19-9 using Raman spectroscopy and surface plasmon resonance (Chung et al., 2006). Although these techniques provide satisfactory specificities and sensitivity in detecting CA 19-9, they are complicated and time-consuming protocols, thus requiring the presence an expert to handle complex and expensive pieces of equipment. In this example, a method is presented for detecting the concentration of CA 19-9 based on negative dielectrophoresis (DEP) spectroscopy.

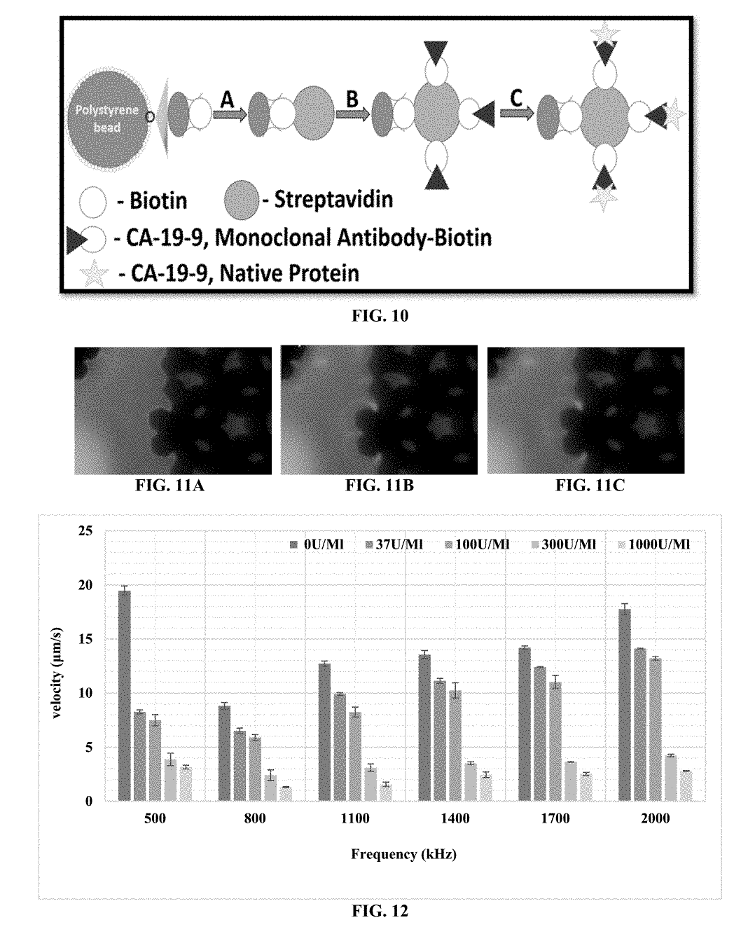

With reference to FIG. 10, the dielectric particle sample was prepared as follows. Commercially-available 740 diameter dielectric particles functionalized with biotin were obtained from Spherotech, Inc. The first step in the preparation of samples was to begin by binding the dielectric particles with the avidin (Vector Labs Inc.). The biotin acts as a conjugate to the protein avidin and forms a strong bond with very high affinity. This process is done first by adding a 3 μL avidin solution into a 10 μL biotin functionalized dielectric particle solution into a centrifuge tube, according to the manufacturer recommendation. The total volume was set to 400 μL by adding 0.01×PBS buffer. Then, the sample was uniformly mixed using a vortex machine and left on a shaker for 20 minutes for the avidin-biotin binding process. After 20 minutes, the tube was centrifuged at 5000 rpm for 14 minutes to remove the unbounded avidin molecules and the buffer.

The next step in preparing the samples was to attach a CA 19-9 antibody to the avidin-biotin functionalized dielectric particles. Each avidin molecule can bind up to 4 biotin molecules. One binding side of each avidin is used to bind each avidin molecule with the dielectric particle and the remaining three avidin binding sites can bind with three biotin-functionalized CA 19-9 antibodies. Three (3) μL of biotin-functionalized CA 19-9 antibody was added to 397 μL of 0.01×PBS buffer. Then this sample was added to the solution containing the avidin-biotin dielectric particles and uniformly mixed using a vortex machine. The sample was kept on a shaker for 20 minutes to allow for the biotin-functionalized CA 19-9 antibody to bind with the avidin molecules of the avidin-biotin dielectric particles. After 20 minutes, the tube was centrifuged at 5000 rpm for 14 minutes to remove the unbounded biotin functionalized CA 19-9 antibody molecules and the buffer.

The next step in preparing the samples was to attach the CA 19-9 antigen to the antibodies on the dielectric particles. For this process, different concentration of CA 19-9 antigen, including 18 U/mL and 37 U/mL, were prepared in a total volume of 400 μL 0.01×PBS buffer. The CA 19-9 antigen was added to the CA 19-9 antibody-bound dielectric particles and mixed uniformly using a vortex machine. Next, the sample was kept on a shaker for 30 mins and then was centrifuged at 5000 rpm for 14 minutes to remove the unbounded CA 19-9 antigen molecules and the buffer. Finally, 200 μL of 0.01×PBS buffer was added to the centrifuge tube and mixed uniformly.

A pearl-shaped interdigitated electrode (PIDE) was designed and drawn to scale in AutoCAD, validated in COMSOL Multiphysics, and fabricated on commercially available glass wafer using photolithography, metal sputtering, and lift-off procedures using 1000 Å thick gold film. The electrode can generate maximum electric field of 1.8×10⁴ V/m with high electric field gradient, which is adequate to produce strong DEP effects on dielectric particles with a few hundred nanometers of diameter. It was observed that the root mean square of the electric field and the electric field gradient produced by this electrode do not vary with the frequency from tens of kHz to several MHz. This ensures the sample is subjected to the same electric field gradient in all frequencies of interest, which makes DEP behavior depend only on the value of Re [K_{CM}(ω)] and, consequently, on the number of analyte molecules attached to the surfaces of the PM.

A clean PIDE electrode, an OMFL600 low power microscope, a Tektronix AFG series function generators, and a custom built green LED lamp illuminating with 45° of incidence were used in the experiment. This angle of incidence reduces the amount of light collected by the camera that does not result from light scattering from the PM. When 10 μL of the assay was pipetted on the surface the electrode, a portion of the light scattered from the antigen bound dielectric particles is collected by the objective of the microscope, resulting in a sharp image of the antigen bound PM. Since the diameter of the antigen bound dielectric particles is of the same order as the wavelength of the green LED (565 nm), the antigen bound dielectric particles appear very bright on a dark background due to Mie scattering. Therefore, this is a label-free method, since no fluorescence markers with affinity to the target molecules need to be used for the transduction. This method avoids the need of a washing procedure to remove the unbound fluorescence markers.

Before the drift velocity due to the DEP force can be measured for any frequency, positive DEP force needs to be applied to attract the dielectric particles to the edges of the electrodes. Then, negative DEP is applied for a frequency in the frequency range of the spectrum used in the experiment. The software sets the function generator to scan through a set of electrical frequencies. For the experiments in this example, the lowest frequency was set to 500 kHz, the frequency step was set to 300 kHz, and the highest frequency was 2 MHz for negative DEP. The peak-to-peak voltage value was set to 10 V.

The time interval for positive DEP was 2000 ms and time interval for negative DEP was set for 80 ms per frequency measurement. In case of positive DEP, the experiment starts with a certain start frequency f₁ that induces positive DEP effect for the specified time interval to collect the dielectric particles near the electrode. Then, the frequency is automatically changed to another frequency f₂ that induces negative DEP effect for the specified time interval and two images are obtained to measure the center of mass of the dielectric particles as they are being repelled from the electrode. Then, the waveform generator changes the frequency back to f₁ to collect the dielectric particles near the electrode. This cycle goes on until f₂ reaches the stop frequency. The frequency switching by the function generator is done automatically by the application disclosed herein.

The spectrum measurement can also be designed to measure the drift velocity due to positive DEP as a function of the frequency. However, positive DEP has a narrower frequency range for the dielectric particles used in this example. Moreover, it was easier to automate the measurement of the drift velocity due to negative DEP because the dielectric particles start drifting from the same region near the edge of the electrodes at the onset of negative DEP.

Initially the function generator automatically sets to a frequency of 10 kHz to establish positive DEP. Low frequency electric fields (<50 kHz) produce positive DEP whereas high frequency electric fields (>250 kHz) produce negative DEP force to the dielectric particles. The positive DEP force resulted in the attraction of the dielectric particles towards the edge of the electrodes. A clear line of dielectric particles can be seen forming at the edge of the electrodes in FIG. 11A, which includes an image taken shortly after negative DEP was applied. FIGS. 11B and 11C were taken after 40 and 80 ms, respectively. Once the dielectric particles form a layer at the edge of the electrode, the software automatically switched the frequency to a preset frequency that produces negative DEP. The negative DEP force pushes the dielectric particles from the edge of the electrode. The repulsion of the dielectric particles are tracked and recorded using the application. The velocity (μm/second) of the dielectric particles repulsed from the electrode as they moved away from the electrode edge was calculated through image processing.

The system tracks the variation of the light intensity along the major axis of the box enclosing the region of interest at different time intervals. The center of mass of the light intensity observed in the image corresponds to the position of the antigen-bound dielectric particles. Two images are used to calculate the speed of repulsion, one captured shortly after negative DEP is applied and the other tens of microseconds later. The center of the mass of the light intensity is calculated for both images and is used to find out the speed of the repulsion due to negative DEP.

A relationship was observed between the speed of repulsion of the dielectric particles functionalized with the CA 19-9 antibodies as a function of both the frequency and the concentrations of the target CA 19-9 antigen. Upon switching to higher frequency electric fields from the application, a clear dependence of the negative DEP spectrum on the concentration of the target CA 19-9 in the sample was observed. While switching the frequency, all other experiment parameters and conditions were maintained constant. The obtained DEP spectroscopy results are shown in FIG. 12. The results were compared to the cutoff levels of CA 19-9 that can be used for the diagnosis and monitoring of the pancreatic cancer (Kamisawa et al., 2016). Using these results, FIG. 12 can be used as the calibration curve for the diagnosis and monitoring of pancreatic cancer at very early stage.

The experiments described in this example demonstrate that negative DEP spectroscopy can be used to accurately detect the concentration of a protein, such as CA 19-9, which is a pancreatic cancer biomarker. The negative DEP spectrum was measured using real-time image processing to detect the velocity in which dielectric particles functionalized with monoclonal antibodies to CA 19-9 are repelled by an interdigitated electrode array due to DEP as a function of the frequency. DEP spectroscopy has sufficient sensitivity to detect the various cutoff levels of CA 19-9 that can enable this method to be used in the diagnosis and in the monitoring of pancreatic cancer. The drift velocity due to negative DEP was calculated for frequency range of 500 kHz to 2000 kHz. The 750 nm diameter dielectric particles were imaged using a side illumination technique to detect the Mie scattering produced by the dielectric particles. The change in the DEP spectrum with the binding of even a small concentration of CA 19-9 to the conjugated antibody binding sites on the dielectric particles arise from the changes the distribution of the ions from the solution close to the dielectric particles surfaces in the presence of the target molecules. Therefore, there is no need to use fluorescent labels conjugated to CA 19-9 to detect the presence of those molecules. Since the measurement using negative DEP spectroscopy is based on the drift velocity of the particles due to DEP as a function of the frequency of the external electric field, this measurement does not depend on the light intensity, the number of dielectric particles, or the sensitivity of the camera, as long as they are sufficient to image the center of mass of the dielectric particles.

### Example 6: SNP Detection Using DEP Spectroscopy

The system of Example 1 was used for further studies. In this example, the application was used to detect DNA molecules, and single nucleotide changes in DNA molecules.

Samples were prepared as follows. First, streptavidin purchased from Vector Labs Inc. was attached to biotinylated dielectric particles that were 750 nm in diameter, which were purchased from Spherotech Inc. Biotin acts as a conjugate to the protein streptavidin and they form a strong bond with very high affinity. This process is done first by adding a 3 μL streptavidin solution into a 10 μL biotinylated PM solution in a centrifuge tube, according to the manufacturer recommendation. The total volume was set to 400 μL by adding 0.01× phosphate-buffered saline (PBS) solution with conductivity 0.01 S/m. Then, the sample was uniformly mixed using a vortex machine and left on a shaker for 20 min for the streptavidin-biotin binding process. After 20 min, the tube was centrifuged at 5000 rpm for 14 min to remove the unbound streptavidin molecules and the buffer.

Next, biotinylated DNA was attached to the dielectric particles. Each streptavidin molecule can bind up to four (4) biotin molecules. One binding site of each streptavidin molecule is used to bind that streptavidin molecule to the biotinylated dielectric particle. The remaining three streptavidin binding sites bind with three (3) biotinylated DNA molecules. Three (3) μL of each biotinylated DNA molecule (The Midland Certified Reagent Company was added into 397 μL of 0.01×Tris-EDTA, according to the manufacturer requirement. The biotinylated DNA sample was added to the solution including streptavidin-biotin dielectric particles and uniformly mixed using a vortex machine. The sample was kept on a shaker for 20 min for the biotinylated DNA to bind with the streptavidin molecules. After 20 min, the tube was centrifuged at 5000 rpm for 14 min to remove the unbound DNA molecules and the buffer. Finally, 200 μL of 0.01×TE buffer was added to the centrifuge tube and mixed uniformly. 10 μL of this sample solution was pipetted on to the microelectrodes and used for each experiment.

The following biotinylated DNA molecules were studied, in which there is a change in last nucleotide of each molecule:

5′-(biotin) TGTTGTGCGA-3′
5′-(biotin) TGTTGTGCGT-3′
5′-(biotin) TGTTGTGCGG-3′
5′-(biotin) TGTTGTGCGC-3′

The following biotinylated DNA molecules were studied, in which there is a change in the second-to-last nucleotide of each molecule:

5′-(biotin) TGTTGTGCAC-3′
5′-(biotin) TGTTGTGCTC-3′
5′-(biotin) TGTTGTGCCC-3

An initial experiment was performed using the following SS DNA sequence: 5′-(biotin) TGTTGTGCGA-3′. The initial frequency is set to 10 kHz to establish low frequency electric field (50 kHz) thus producing positive DEP and the sweeping frequency from 500 kHz to 2 MHz establishing higher frequency electric field (>250 kHz) producing positive DEP.

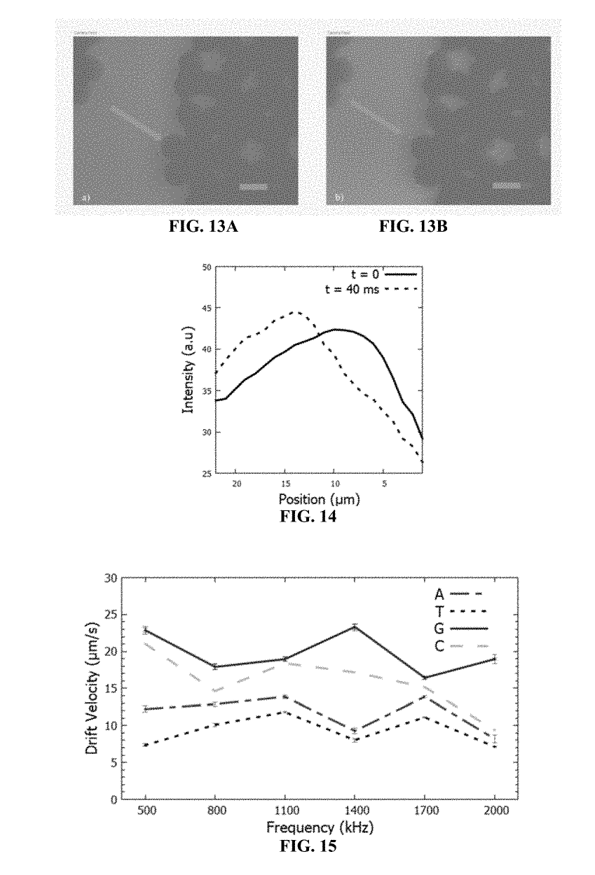

With reference to FIGS. 13A and 13B, the interdigitated electrode is visible as a darker region in the picture. The bright layer visible on the edge of the electrode is formed by the accumulation of the dielectric particles. For 60 seconds prior to the image in FIG. 13A, a 10 V peak-to-peak electric field at 10 kHz, which produces positive DEP, was applied to the electrodes. Right after that, the frequency of the electric field increased to 500 kHz, producing negative DEP. As the frequency is changed to induce negative DEP the antigen bound dielectric particles are repelled from the electrode. FIG. 13B depicts the repulsion of the dielectric particles away from the electrodes due to negative DEP at 40 ms. Using this concept, system can calculate the drift velocity of the dielectric particle layer proportional to the DEP force using the image processing technique disclosed herein.

With further reference to FIGS. 13A and 13B, the region of interest is indicated by the arrows. The region of interest tracks the variation of the light intensity with respect to the frequency applied at the negative DEP. FIG. 14 shows the light intensity of the images, as a function of position, captured shortly after the application of negative DEP and 40 ms later. The average location of the dielectric particles can be calculated using the center of mass of the light intensity curves. The difference in the curve results in the change of speed of repulsion due to the application of negative DEP. The formula used to calculate the center of mass of the light intensity curve is given by

$$Center of Mass = \frac{{\sum }_{i = 1}^{N} {I}_{i} {x}_{i}}{{\sum }_{i = 1}^{N} {I}_{i}}$$

where I is the value of the light intensity and x is the pixel position.

Experiments were performed using the dielectric particles attached to the following biotinylated DNA molecules, in which there is a change in last nucleotide of each molecule: 5′-(biotin) TGTTGTGCGA-3′, 5′-(biotin) TGTTGTGCGT-3′, 5′-(biotin) TGTTGTGCGG-3′, and 5′-(biotin) TGTTGTGCGC-3′. The experiments were run for fourteen frequencies for higher resolution of the spectrum. All the other parameters and the conditions were maintained constant throughout the experiments.

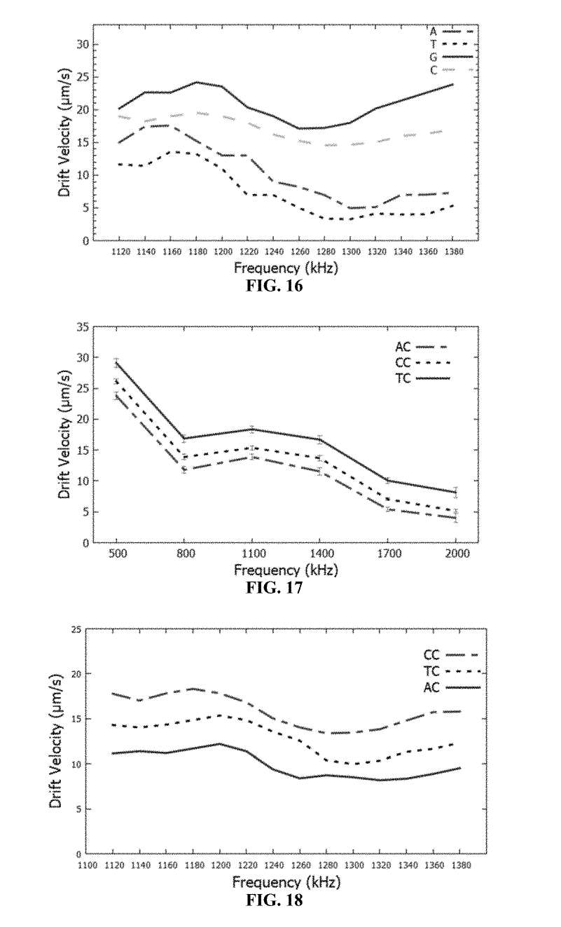

A relationship was observed between the repulsion or drift velocity of the dielectric particles functionalized with the different SS DNA molecules as a function of frequency. A clear dependence was observed for the negative DEP spectrum on the change in the nucleotide sequence. Indeed, the changes of just a single nucleotide resulted in significant changes in drift velocity as between the tested SS DNA molecules. The resulting spectroscopy is as shown in FIG. 15. The error bars with standard deviation obtained in six measurements is a clear indication of confidence interval for each measurement. There is no overlap between the confidence intervals of each curves which eliminates the need of repeatability. FIG. 16 depicts the higher resolution spectrum for change in last nucleotide.

Experiments were also performed using the dielectric particles attached to the following biotinylated DNA molecules, in which there is a change in the second-to-last nucleotide of each molecule: 5′-(biotin) TGTTGTGCAC-3′, 5′-(biotin) TGTTGTGCTC-3′, and 5′-(biotin) TGTTGTGCCC-3. The experiments were run for fourteen frequencies for higher resolution of the spectrum. All the other parameters and the conditions were maintained constant throughout the experiments.

A relationship was observed between the repulsion or drift velocity of the dielectric particles functionalized with the different SS DNA molecules as a function of frequency. A clear dependence was observed for the negative DEP spectrum on the change in the nucleotide sequence. Indeed, the changes of just a single nucleotide resulted in significant changes in drift velocity as between the tested SS DNA molecules. The resulting spectroscopy is as shown in FIG. 17. The error bars with standard deviation obtained in six measurements is a clear indication of confidence interval for each measurement. There is no overlap between the confidence intervals of each curves which eliminates the need of repeatability. FIG. 18 depicts the higher resolution for the same range for the change in second last nucleotide.

In view of the results shown in FIGS. 15-18, this label free method can be used as the transduction mechanism for single nucleotide polymorphism (SNP) detection.

### Example 7: Electrode

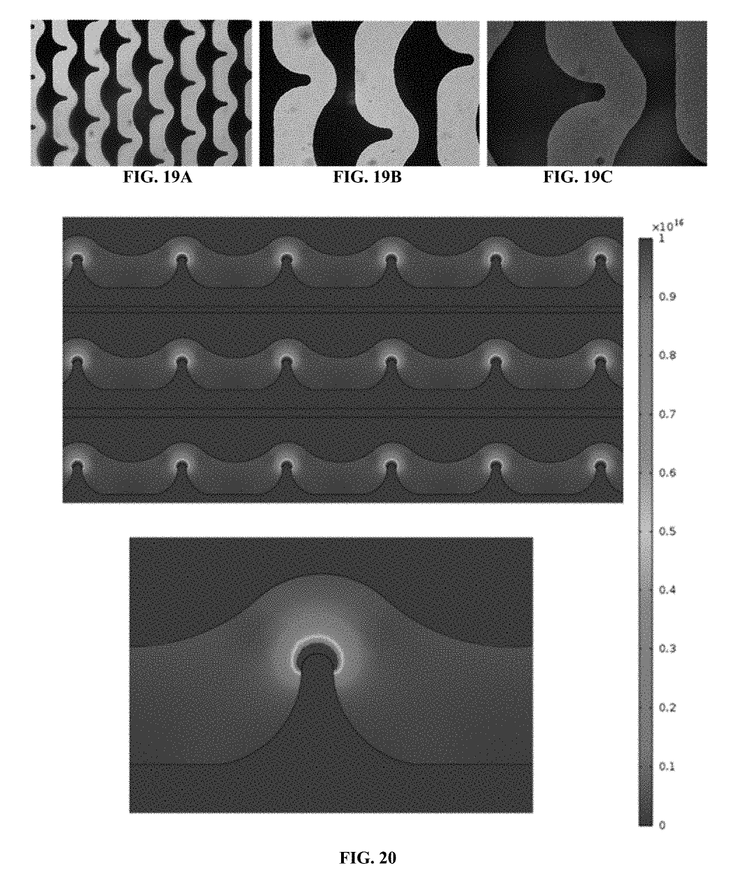

The design of the electrode was drawn to scale using AutoCAD and then its electric field gradient was validated using COMSOL Multiphysics. The images of the electrode at different magnifications of 10×, 25× and 40× are shown in FIGS. 19A-19C.

The electrode was fabricated on a commercially-available glass wafer with standard fabrication procedures involving photolithography, metal sputtering and lift-off procedures using 1000 Å thick gold film in the microfabrication facilities at North Dakota State University. Using COMSOL the gradient of the electric field intensity was verified to be 1×1016 V2/m3. The simulation results using COMSOL Multiphysics is as shown in FIG. 20.

No variation was observed on the root mean square of the electric field and the electric field gradient produced by this electrode with respect to a frequency ranging from several kHz to a few MHz. One of the drawbacks of using the previously used pearl Shaped Interdigitated Electrode is that the polystyrene microspheres (PM) concentration varies for every cycle of positive and negative DEP. This is because there is gradient applied on each side, thus those PM which experiences the negative DEP may not be repelled back for the positive DEP completely. The new electrode is designed in such a way that on one side of the array there is the electrode edge and on the opposite side, there is a concave structure with no sharp edges. With no sharp edges, the concave structure would have very minimal or no DEP acting on it. With the new electrode the PM that experience the positive DEP would attract on the edge of the electrode and during the negative DEP, it will be repelled away from the edge of the electrode. In the next cycle of frequency, the same set of PMs will be attracted back to the edge of the electrode as there is no or very minimal gradient on the concave structure. This made the lab experiments more robust, helped maintain uniformity and it also optimized the experiment time.

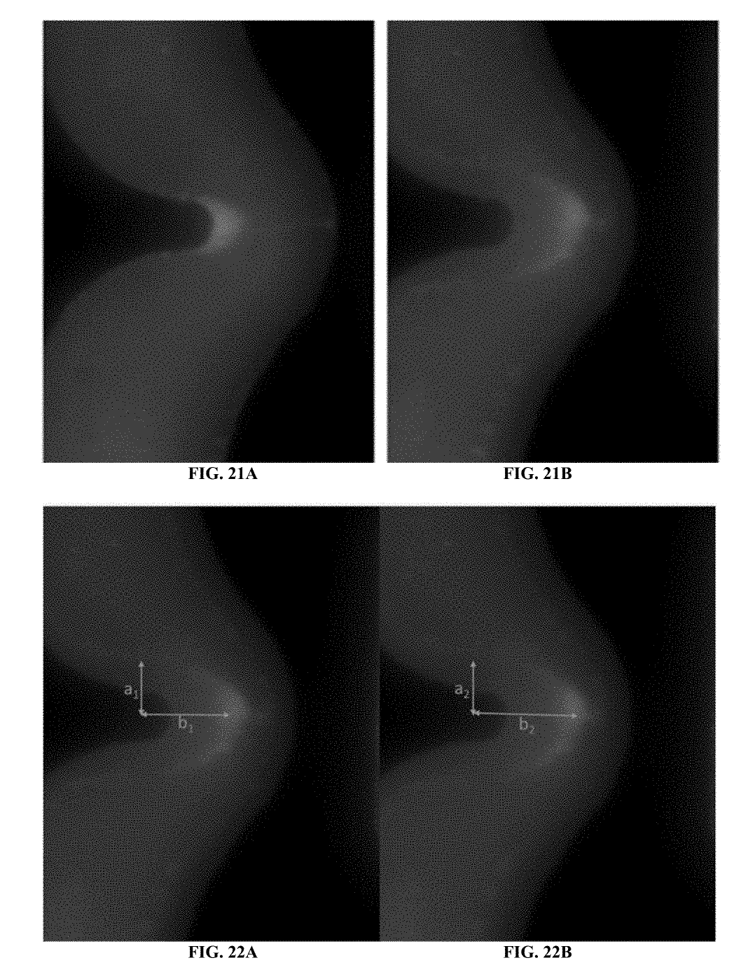

The application of positive DEP attracted the PM on the edge of the electrodes and the application of negative DEP repelled the PM away from the electrode edge. The FIG. 21A shows the PM lining on the edge of the electrode with positive DEP acting on it up to 0 ms, when negative DEP is applied. FIG. 21B shows the distribution of the PM due to negative DEP at 40 ms.

The distribution of the PM traces the shape of an ellipse. The PM accumulation and repulsion force are higher at the major axis (b1 and b2 as shown in FIG. 22) in comparison to the accumulation and repulsion at the minor axis (a1 and a2 as shown in FIG. 22). This is explained in FIG. 22. The software developed using Microsoft foundation classes in Visual C++ for Microsoft operating system measures the drift velocity of dielectric particles due to DEP as a function of the frequency of the electric field applied to the interdigitated electrode. In the previous version, the raster scan happens for each row and columns of the region of interest. With the electrode the PM tracing the shape of an ellipse the software scan pattern will be radial.

A change in the negative DEP force applied to the PM was observed with respect to change in a single stranded (SS) DNA sequence and frequency of the electric field produced by new interdigitated microelectrodes. The drift velocity of the PM functionalized to a set of SS DNA of different sequence at a frequency range of 500 kHz to 2000 kHz in steps of 300 kHz. The drift velocity was calculated using a custom-made automated software using real-time image processing technique that captures the Mie scattering of the Pm due to the side illumination.

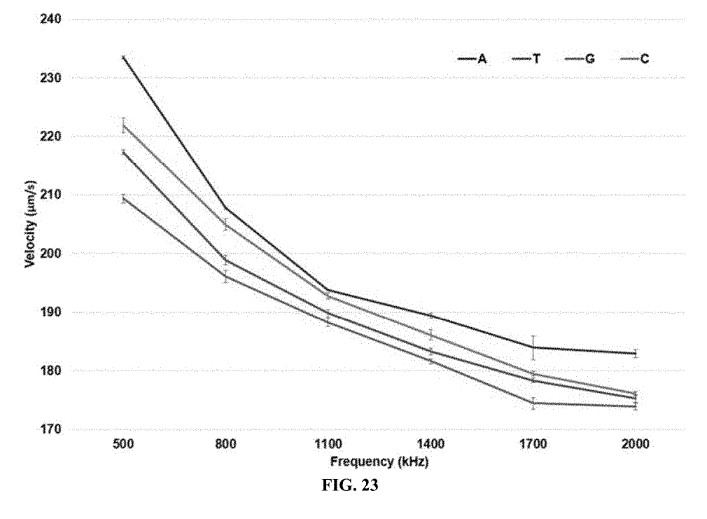

The assay had Biotinylated DNA attached to the biotinylated PM+Streptavidin. Each Streptavidin molecule can bind up to 4 biotin molecules. One binding site of each Streptavidin molecule is used to bind that Streptavidin molecule with the PM. The remaining three Streptavidin binding sites bind with three biotinylated DNA. This sample was used with 0.01×TE buffer. A relationship was observed between the drift velocity of the PM functionalized with SS DNA as a function of frequency with the change in the single nucleotide. The SS DNA sequence used for this is 5′-(biotin) TGTTGTGCGA-3′ with the last nucleotide being varied. FIG. 23 shows the negative DEP spectrum curves for the change in the last nucleotide sequence for frequency varying from 500 kHz to 2000 kHz in steps of 300 kHz. The error bars show the confidence interval in each individual measurement that was calculated using six measurements per frequency.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. E. Altinagac, S. S. Ozcan, Y. Genc, H. Kizil, and L. Trabzon, “Biological particle manipulation: An example of Jurkat enrichment,” 2015 IEEE 10th Int. Conf. Nano/Micro Eng. Mol. Syst. NEMS 2015, vol. 10, pp. 25-27, 2015.
2. Altintas, Zeynep, Yildiz Uludag, Yasar Gurbuz, and Ibtisam E. Tothill. 2011. “Surface Plasmon Resonance Based Immunosensor for the Detection of the Cancer Biomarker Carcinoembryonic Antigen.” Talanta 86:377-83.
3. D. J. Bakewell, J. Bailey, D. Holmes, “Real-time dielectrophoretic signaling and image quantification methods for evaluating electrokinetic properties of nanoparticles,” Electrophoresis 36(13), 1443-1450 (2015).
4. L. M. Broche, et al., “Early detection of oral cancer—Is dielectrophoresis the answer?,” Oral Oncol. 43(2), 199-203 (2007).
5. W. S. Bush and J. H. Moore, “Chapter 11: Genome-Wide Association Studies,” PLoS Comput. Biol., vol. 8, no. 12, 2012.
6. H. Cai, T. M. H. Lee, I. M. Hsing, “Label-free protein recognition using an aptamer-based impedance measurement assay,” Sensors Actuators, B Chem. 114(1), 433-437 (2006).
7. Cai, Hong, Thomas Ming Hung Lee, and I. Ming Hsing. 2006. “Label-Free Protein Recognition Using an Aptamer-Based Impedance Measurement Assay.” Sensors and Actuators, B: Chemical 114(1):433-37.
8. Camacho-alanis, Fernanda and Alexandra Ros. 2015. “Protein Dielectrophoresis and the Link to Dielectric Properties.” Bioanalysis 7(3):353-71.
A. Castellanos et al., “Electrohydrodynamics and dielectrophoresis in microsystems: scaling laws,” J. Phys. D. Appl. Phys. 36(20), 2584-2597, IOP Publishing (2003) [doi:10.1088/0022-3727/36/20/023].
9. Castellanos, A., A. Ramos, A. Gonzalez, N. G. Green, and H. Morgan. 2003. “Electrohydrodynamics and Dielectrophoresis in Microsystems: Scaling Laws.” Journal of Physics D: Applied Physics 36(20):2584-97.
10. Chan, Alison et al. 2014. “Validation of Biomarkers That Complement CA19.9 in Detecting Early Pancreatic Cancer.” Clinical Cancer Research 20(22):5787-95.
11. C. Chen et al., “Methods for Identifying SNP Interactions: A Review on Variations of Logic Regression, Random Forest and Bayesian Logistic . . . Methods for Identifying SNP Interactions: A Review on Variations of Logic Regression, Random Forest and Bayesian Logistic Reg,” Ieee/Acm Trans. Comput. Biol. Bioinformatics, vol. 8, no. August 2017, pp. 1580-1591, 2011.
12. H. Chen, et al., “Protein chips and nanomaterials for application in tumor marker immunoassays,” in Biosensors and Bioelectronics 24(12), pp. 3399-3411 (2009).
13. H.-H. Chen, et al., “High-purity separation of cancer cells by optically induced dielectrophoresis,” J. Biomed. Opt. 19(4), 45002 (2014).
14. Chen, Hsiu-Hsiang et al. 2014. “High-Purity Separation of Cancer Cells by Optically Induced Dielectrophoresis.” Journal of Biomedical Optics 19(4):45002.
15. Chen, Hui et al. 2009. “Protein Chips and Nanomaterials for Application in Tumor Marker Immunoassays.” Biosensors and Bioelectronics 24(12):3399-3411.
16. Y. H. Cheng, “A Novel Teaching-Learning-Based Optimization for Improved Mutagenic Primer Design in Mismatch PCR-RFLP SNP Genotyping,” IEEE/ACM Trans. Comput. Biol. Bioinforma., vol. 13, no. 1, pp. 86-98, 2016.
17. Y. H. Cheng, C. N. Kuo, and C. M. Lai, “Effective natural PCR-RFLP primer design for SNP genotyping using teaching-learning-based optimization with elite strategy,” IEEE Trans. Nanobioscience, vol. 15, no. 7, pp. 657-665, 2016.
18. B. V. Chikkaveeraiah, et al., Electrochemical immunosensors for detection of cancer protein biomarkers, in ACS Nano 6(8), pp. 6546-6561, American Chemical Society (2012).
19. Chikkaveeraiah, Bhaskara V., Ashwinkumar A. Bhirde, Nicole Y. Morgan, Henry S. Eden, and Xiaoyuan Chen. 2012. “Electrochemical Immunosensors for Detection of Cancer Protein Biomarkers.” ACS Nano 6(8):6546-61.
20. Chu, Chia-Ho et al. 2017. “Beyond the Debye Length in High Ionic Strength Solution: Direct Protein Detection with Field-Effect Transistors (FETs) in Human Serum.” Scientific Reports 7(1):5256.
21. Chung, J. W., Bernhardt, R., Pyun, J. C., 2006. Additive assay of cancer marker CA 19-9 by SPR biosensor. Sensors Actuators, B Chem. 118, 28-32. doi:10.1016/j.snb.2006.04.015
22. Cui, Li, David Holmes, and Hywel Morgan. 2001. “The Dielectrophoretic Levitation and Separation of Latex Beads in Microchips.” Electrophoresis 22(18):3893-3901.
23. J. S. Daniels, N. Pourmand, “Label-free impedance biosensors: Opportunities and challenges,” in Electroanalysis 19(12), pp. 1239-1257, NIH Public Access (2007).
24. Daniels, Jonathan S. and Nader Pourmand. 2007. “Label-Free Impedance Biosensors: Opportunities and Challenges.” Electroanalysis 19(12):1239-57.
25. M. A. Daubert, A. Jeremias, “The utility of troponin measurement to detect myocardial infarction: Review of the current findings,” Vasc. Health Risk Manag. 6(1), 691-699, Dove Press (2010).
26. Del Villano, B. C., Brennan, S., Brock, P., Bucher, C., Liu, V., McClure, M., Rake, B., Space, S., Westrick, B., Schoemaker, H., Zurawski, V. R., 1983. Radioimmunometric assay for a monoclonal antibody-defined tumor marker, CA 19-9. Clin. Chem. 29, 549-552.
27. H. Donis-Keller et al., “A genetic linkage map of the human genome,” Cell, vol. 51, no. 2, pp. 319-337, 1987.
28. Duffy, M. J., Sturgeon, C., Lamerz, R., Haglund, C., Holubec, V. L., Klapdor, R., Nicolini, A., Topolcan, O., Heinemann, V., 2009. Tumor markers in pancreatic cancer: A European Group on Tumor Markers (EGTM) status report. Ann. Oncol. 21, 441-447. doi:10.1093/annonc/mdp332
29. F. J. Gella, J. Serra, J. Gener, “Latex Agglutination Procedures in Immunodiagnosis,” Pure Appl. Chem. 63(8), 1131-1134 (1991).
30. Gella, F. J., J. Serra, and J. Gener. 1991. “Latex Agglutination Procedures in Immunodiagnosis.” Pure and Applied Chemistry 63(8):1131-34. Retrieved (isi:A1991FX06300011).
31. Green, Nicolas G. and Hywel Morgan. 1999. “Dielectrophoresis of Submicrometer Latex Spheres. 1. Experimental Results.” The Journal of Physical Chemistry B 103(1):41-50.
32. F. D. Gudagunti, L. Velmanickam, D. Nawarathna, and I. T. Lima, “Label-free biosensing method for the detection of a pancreatic cancer biomarker based on dielectrophoresis spectroscopy,” Chemosensors, vol. 6, no. 3, 2018.
33. Gudagunti, Fleming Dackson, Logeeshan Velmanickam, Dharmakeerthi Nawarathna, and Ivan T. Lima Jr. 2017. “Label-Free DNA Identification Using Light Scattering from Microbeads and Dielectrophoresis Spectroscopy.” 595-96.
34. F. D. Gudagunti, L. Velmanickam, D. Nawarathna, and I. T. Lima, “Label-free DNA identification using light scattering from microbeads and dielectrophoresis spectroscopy,” in 30th Annual Conference of the IEEE Photonics Society, IPC 2017, 2017, vol. 2017—Janua.
35. Gupta, Shalini, Peter K. Kilpatrick, Elizabeth Melvin, and Orlin D. Velev. 2012. “On-Chip Latex Agglutination Immunoassay Readout by Electrochemical Impedance Spectroscopy.” Lab on a Chip 12(21):4279-86.
36. J. He and A. Zelikovsky, “Informative SNP selection methods based on SNP prediction,” IEEE Trans. Nanobioscience, vol. 6, no. 1, pp. 60-67, 2007.
37. Henry, N. L., Hayes, D. F., 2012. Cancer biomarkers. Mol. Oncol. 6, 140-146.
38. G. L. Hornyak, et al., Fundamentals of Nanotechnology, CRC Press (2008).
39. Jedinak, Andrej et al. 2015. “Novel Non-Invasive Biomarkers That Distinguish between Benign Prostate Hyperplasia and Prostate Cancer.” BMC Cancer 15(1):259.
40. Jones, Thomas B. 2003. “Basic Theory of Dielectrophoresis and Electrorotation.” IEEE Engineering in Medicine and Biology Magazine 22(6):33-42.
41. Jubery, Talukder Z., Soumya K. Srivastava, and Prashanta Dutta. 2014. “Dielectrophoretic Separation of Bioparticles in Microdevices: A Review.” Electrophoresis 35(5):691-713.
42. Kamisawa, T., Wood, L. D., Itoi, T., Takaori, K., 2016. Pancreatic cancer. Lancet (London, England) 388, 73-85. doi:10.1016/S0140-6736(16)00141-0
43. T. Kawabata and M. Washizu, “Dielectrophoretic detection of molecular bindings,” IEEE Trans. Ind. Appl., vol. 37, no. 6, pp. 1625-1633, 2001.
44. K. Khoshmanesh, S. Nahavandi, S. Baratchi, A. Mitchell, and K. Kalantar-zadeh, “Dielectrophoretic platforms for bio-microfluidic systems,” Biosens. Bioelectron., vol. 26, no. 5, pp. 1800-1814, 2011.
45. S. A. M. Kirmani, et al., “Dielectrophoretic Spectroscopy Using a Microscopic Electrode Array,” In Proc. SPIE Photonics West 2017, pp 100680Z 1-13, 2017.
46. S. A. M. Kirmani, F. D. Gudagunti, L. Velmanickam, D. Nawarathna, and I. T. Lima, “Negative dielectrophoresis spectroscopy for rare analyte quantification in biological samples,” J. Biomed. Opt., vol. 22, no. 3, 2017.
47. P. Y. Kwok and X. Chen, “Detection of single nucleotide polymorphisms.,” Curr Issues Mol Biol, vol. 5, no. 2, pp. 43-60, 2003.
48. T. LaFramboise, “Single nucleotide polymorphism arrays: A decade of biological, computational and technological advances,” Nucleic Acids Res., vol. 37, no. 13, pp. 4181-4193, 2009.
49. Li, M., W. H. Li, J. Zhang, G. Alici, and W. Wen. 2014. “A Review of Microfabrication Techniques and Dielectrophoretic Microdevices for Particle Manipulation and Separation.” Journal of Physics D: Applied Physics 47(6):63001.
50. R. Li et al., “SNP detection for massively parallel whole-genome resequencing.-ppt,” Genome Res., vol. 19, no. 6, pp. 1124-32, 2009.
51. Lynch, Brian P., Al M. Hilton, and Garth J. Simpson. 2006. “Nanoscale Dielectrophoretic Spectroscopy of Individual Immobilized Mammalian Blood Cells.” Biophysical Journal 91(7):2678-86.
52. B. P. Lynch, A. M. Hilton, and G. J. Simpson, “Nanoscale dielectrophoretic spectroscopy of individual immobilized mammalian blood cells.,” Biophys. J. 91(7), 2678-2686, The Biophysical Society (2006).
53. B. Maher, “Personal genomes: The case of the missing heritability,” Nature, vol. 456, no. 7218, pp. 18-21, 2008.
54. K. Martin, et al., “Troponin elevations in patients with chronic cardiovascular disease: An analysis of current evidence and significance.,” Ann. Card. Anaesth. 19(2), 321-327 (2016).
55. Meyer, Martin H. F., Markus Hartmann, and Michael Keusgen. 2006. “SPR-Based Immunosensor for the CRP Detection—A New Method to Detect a Well Known Protein.” Biosensors and Bioelectronics 21:1987-90.
56. J. A. Molina-Bolivar, F. Galisteo-Gonzalez, “Latex Immunoagglutination Assays,” J. Macromol. Sci. Part C Polym. Rev. 45(1), 59-98, Taylor & Francis Group (2005).
57. Morrow, David A. et al. 2007. “National Academy of Clinical Biochemistry Laboratory Medicine Practice Guidelines: Clinical Characteristics and Utilization of Biochemical Markers in Acute Coronary Syndromes.” Clinical Chemistry 574:552-74.
58. Muniraj, T., Jamidar, P. A., Aslanian, H. R., 2013. Pancreatic cancer: A comprehensive review and update. Disease-a-Month 59, 368-402. doi:10.1016/j.disamonth.2013.08.001
59. F. Negr and A. Cavallini, “Effect of dielectrophoretic forces on nanoparticles,” IEEE Trans. Dielectr. Electr. Insul., vol. 24, no. 3, pp. 1708-1717, 2017.
60. O'Brien, Darragh P. et al. 2015. “Serum CA19-9 Is Significantly Upregulated up to 2 Years before Diagnosis with Pancreatic Cancer: Implications for Early Disease Detection.” Clinical Cancer Research 21(3):622-31.
61. Panackal, Anil A., John P. Dekker, Michael Proschan, Andrea Beri, and Peter R. Williamson. 2014. “Enzyme Immunoassay versus Latex Agglutination Cryptococcal Antigen Assays in Adults with Non-HIV-Related Cryptococcosis.” Journal of Clinical Microbiology 52(12):4356-58.
62. Papagiakoumou, Eirini, Dorel Pietreanu, Mersini I. Makropoulou, Eugenia Kovacs, and Alexander A. Serafetinides. 2006. “Evaluation of Trapping Efficiency of Optical Tweezers by Dielectrophoresis.” Journal of Biomedical Optics 11(1):14035
63. Passerini, R., Cassatella, M. C., Boveri, S., Salvatici, M., Radice, D., Zorzino, L., Galli, C., Sandri, M. T., 2012. The pitfalls of CA19-9: Routine testing and comparison of two automated immunoassays in a reference oncology center. Am. J. Clin. Pathol. 138, 281-287.
64. Pawula, Maria, Zeynep Altintas, and Ibtisam E. Tothill. 2016. “SPR Detection of Cardiac Troponin T for Acute Myocardial Infarction.” Talanta 146:823-30.
65. G. Pesce, et al., “Simultaneous measurements of electrophoretic and dielectrophoretic forces using optical tweezers,” Opt. Express 23(7), 9363, Optical Society of America (2015).
66. R. Pethig, “Dielectrophoresis: Status of the theory, technology, and applications,” Biomicrofluidics 4(2), 22811, American Institute of Physics (2010).
67. Pethig, Ronald. 2010. “Dielectrophoresis: Status of the Theory, Technology, and Applications.” Biomicrofluidics 4(2):1-35.
68. R. Pethig, S. Smith, Introductory Bioelectronics: For Engineers and Physical Scientists, in Introductory Bioelectronics: For Engineers and Physical Scientists, John Wiley & Sons (2013).
69. P. M. Pilarski, C. J. Backhouse, “A method for cytometric image parameterization.,” Opt. Express 14(26), 12720-12743 (2006).
70. Poruk, K. E., Gay, D. Z., Brown, K., Mulvihill, J. D., Boucher, K. M., Scaife, C. L., Firpo, M. A., Mulvihill, S. J., 2013. The clinical utility of CA 19-9 in pancreatic adenocarcinoma: diagnostic and prognostic updates. Curr. Mol. Med. 13, 340-51. doi:10.2174/1566524011313030003
71. Prensner, J. R., M. A. Rubin, J. T. Wei, and A. M. Chinnaiyan. 2012. “Beyond PSA: The Next Generation of Prostate Cancer Biomarkers.” Science Translational Medicine 4(127).
72. Puiu, Mihaela and Camelia Bala. 2016. “SPR and SPR Imaging: Recent Trends in Developing Nanodevices for Detection and Real-Time Monitoring of Biomolecular Events.” Sensors 16(870):1-15.
73. Qiu, Wanwei et al. 2015. “Carbon Nanotube-Based Lateral Flow Biosensor for Sensitive and Rapid Detection of DNA Sequence.” Biosensors & Bioelectronics 64:367-72.
74. J. Ramoń-Azcón, T. Yasukawa, and F. Mizutani, “Sensitive and spatially multiplexed detection system based on dielectrophoretic manipulation of DNA-encoded particles used as immunoreactions platform,” Anal. Chem., vol. 83, no. 3, p. 1053, 2011.
75. Ramos, A., H. Morgan, N. G. Green, and A. Castellanos. 1999. “Ac Electrokinetics: A Review of Forces in Microelectrode Structures.” Journal of Physics D: Applied Physics 31(18):2338-53.
76. Robertson et al., “Discriminating a Single Nucleotide Difference for Enhanced miRNA,” Langmuir, Vol. 31(36), pp 9943-995, 2015.
77. Rodriguez, Marcela C., Abdel-Nasser Kawde, and Joseph Wang. 2005. “Aptamer Biosensor for Label-Free Impedance Spectroscopy Detection of Proteins Based on Recognition-Induced Switching of the Surface Charge.” Chemical Communications (34):4267.
78. Rosenfeld, Nitzan et al. 2008. “MicroRNAs Accurately Identify Cancer Tissue Origin.” Nature Biotechnology 26(4):462-69.
79. Rusling, James F., Challa V Kumar, J. Silvio Gutkind, and Vyomesh Patel. 2010. “Measurement of Biomarker Proteins for Point-of-Care Early Detection and Monitoring of Cancer.” The Analyst 135(10):2496-2511.
80. N. R. Shanmugam, S. Muthukumar, S. Prasad, “Ultrasensitive and low-volume point-of-care diagnostics on flexible strips—a study with cardiac troponin biomarkers,” Sci. Rep. 6, 33423, Nature Publishing Group (2016).
81. C. Shekarchi, et al., “Avidin-biotin latex agglutination assay for detection of antibodies to viral antigens,” J. Clin. Microbiol. 26(5), 954-956 (1988).
82. Shekarchi, I. C., D. A. Fuccillo, J. L. Sever, and D. M. Madden. 1988. “Avidin-Biotin Latex Agglutination Assay for Detection of Antibodies to Viral Antigens.” Journal of Clinical Microbiology 26(5):954-56.
83. Y. Shen et al., “A SNP discovery method to assess variant allele probability from next-generation resequencing data,” Genome Res., vol. 20, no. 2, pp. 273-280, 2010.
84. Michael Snyder, Jiang Du, and Mark Gerstein, “Personal genome sequencing: current approaches and challenges,” Genes & Development, vol. 24(5), pp. 423-431, 2010.
85. H. Song, D. J. Bennett, “A semi-analytical approach using artificial neural network for dielectrophoresis generated by parallel electrodes,” J. Electrostat. 68(1), 49-56 (2010).
86. Suehiro, et al., “Quantitative estimation of biological cell concentration suspended in aqueous medium by using dielectrophoretic impedance measurement method,” J. Phys. D. Appl. Phys. 32(21), 2814-2820, IOP Publishing (1999).
87. Takalkar, Sunitha et al. 2016. “Gold Nanoparticle Coated Silica Nanorods for Sensitive Visual Detection of microRNA on a Lateral Flow Strip Biosensor.” Analytical Sciences 32(6):617-22.

A. K. Talukder, S. Gandham, H. A. Prahalad, and N. P. Bhattacharyya, “Cloud-MAQ: The Cloud-enabled Scalable Whole Genome Reference Assembly Application,” 2010 7th Int. Conf. Wirel. Opt. Commun. Networks, WOCN2010, pp. 0-4, 2010.
88. Thévenot, Daniel R., Klara Toth, Richard A. Durst, and George S. Wilson. 2001. “Electrochemical Biosensors: Recommended Definitions and Classification.” Biosensors and Bioelectronics 16(1-2): 121-31.
89. T. T. Toma, Z. Williams, J. Dawson, and D. Adjeroh, “What can one chromosome tell us about human biogeographical ancestry?,” Proc.—2017 IEEE Int. Conf. Bioinforma. Biomed. BIBM 2017, vol. 2017—Janua, pp. 188-193, 2017.
90. M. Varshney, et al., “A label-free, microfluidics and interdigitated array microelectrode-based impedance biosensor in combination with nanoparticles immunoseparation for detection of Escherichia coli O157:H7 in food samples,” Sensors Actuators, B Chem. 128(1), 99-107 (2007).
91. Varshney, Madhukar, Yanbin Li, Balaji Srinivasan, and Steve Tung. 2007. “A Label-Free, Microfluidics and Interdigitated Array Microelectrode-Based Impedance Biosensor in Combination with Nanoparticles Immunoseparation for Detection of Escherichia Coli O157:H7 in Food Samples.” Sensors and Actuators, B: Chemical 128(1):99-107.
92. Velmanickam, Logeeshan, Darrin Laudenbach, and Dharmakeerthi Nawarathna. 2016. “Dielectrophoretic Label-Free Immunoassay for Rare-Analyte Quantification in Biological Samples.” Physical Review E—Statistical, Nonlinear, and Soft Matter Physics 94(4):1-6.
93. Velmanickam, Logeeshan and Keerthi Nawarathna. 2016. “Dielectrophoretic Cell Isolation in Microfluidics Channels for High-Throughput Biomedical Applications.” IEEE International Conference on Electro Information Technology 2016:302-6.
94. M. Viefhues and R. Eichhorn, “DNA dielectrophoresis: Theory and applications a review,” Electrophoresis, vol. 38, no. 11, pp. 1483-1506, March 2017.
95. M. Viefhues, J. Regtmeier, and D. Anselmetti, “Fast and continuous-flow separation of DNA-complexes and topological DNA variants in microfluidic chip format,” Analyst, vol. 138, no. 1, pp. 186-196, 2012.
96. M. T. Wei, J. Junio, D. H. Ou-Yang, “Direct measurements of the frequency-dependent dielectrophoresis force,” Biomicrofluidics 3(1), 1-8 (2009).
97. Weng, Ping You, I. An Chen, Che Kai Yeh, Pin Yi Chen, and Jia Yang Juang. 2016. “Size-Dependent Dielectrophoretic Crossover Frequency of Spherical Particles.” Biomicrofluidics 10(1):1-11.
98. Wu, Jie, Zhifeng Fu, Feng Yan, and Huangxian Ju. 2007. “Biomedical and Clinical Applications of Immunoassays and Immunosensors for Tumor Markers.” TrAC—Trends in Analytical Chemistry 26(7):679-88.
99. Xiao, Hua et al. 2012. “Proteomic Analysis of Human Saliva From Lung Cancer Patients Using Two-Dimensional Difference Gel Electrophoresis and Mass Spectrometry.” Molecular & Cellular Proteomics 11(2):M111.012112.
100. Xu, Hui et al. 2014. “Gold-Nanoparticle-Decorated Silica Nanorods for Sensitive Visual Detection of Proteins.” Analytical Chemistry 86(15):7351-59. Retrieved May 26, 2017 (http://pubs.acs.org/doi/abs/10.1021/ac502249f).
101. Yafouz, Bashar, Nahrizul Adib Kadri, and Fatimah Ibrahim. 2014. “Dielectrophoretic Manipulation and Separation of Microparticles Using Microarray Dot Electrodes.” Sensors (Basel, Switzerland) 14(4):6356-69.
102. N. N. Yahya, N. A. Aziz, M. R. Buyong, and B. Y. Majlis, “Size-based particles separation utilizing dielectrophoresis technique,” Proc. 2017 IEEE Reg. Symp. Micro Nanoelectron. RSM 2017, pp. 10-13, 2017.
103. Zhu, H., Fan, G. C., Abdel-Halim, E. S., Zhang, J. R., Zhu, J. J., 2016. Ultrasensitive photoelectrochemical immunoassay for CA19-9 detection based on CdSe@ZnS quantum dots sensitized TiO2NWs/Au hybrid structure amplified by quenching effect of Ab2@V2+conjugates. Biosens. Bioelectron. 77, 339-346. doi:10.1016/j.bios.2015.09.051
104. Zou, Zhiwei, Junhai Kai, Michael J. Rust, Jungyoup Han, and Chong H. Ahn. 2007. “Functionalized Nano Interdigitated Electrodes Arrays on Polymer with Integrated Microfluidics for Direct Bio-Affinity Sensing Using Impedimetric Measurement.” Sensors and Actuators, A: Physical 136(2):518-26.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A system for determining an effect of a non-uniform electric field on a dielectric particle, comprising:
(a) a computer;
(b) a pair of electrodes for generating the non-uniform electric field;
(c) a function generator in communication with the computer and the electrodes for changing the frequency of the electric field; and
(d) a camera in communication with a microscope and the computer for capturing a series of images of the dielectric particle in the non-uniform electric field;
(c) wherein the computer is programmed to detect changes between the images in the series of images to determine the effect of the non-uniform electric field on the dielectric particle.

2. The system of claim 1, wherein the computer is programmed to communicate with the function generator to induce positive dielectrophoresis (DEP).

3. The system of claim 1, wherein the computer is programmed to communicate with the function generator to induce negative DEP.

4. The system of claim 1, wherein the computer is programmed to communicate with the function generator to start, stop, and step frequency; to control peak to peak voltage; to control a time interval for positive DEP frequency, and/or to control a time interval for negative DEP frequency.

5. The system of claim 1, wherein the computer is programmed to communicate with the function generator provide a start frequency that induces positive DEP until such time as the positive DEP force results in the attraction of the dielectric particle to the electrode, at which time the computer is programmed to communicate with the function generator to change automatically to another frequency that induces negative DEP.

6. The system of claim 5, wherein the computer is further programmed to generate a spectrum of light intensity as a function of position for the dielectric particle.

7. The system of claim 5, wherein the computer is further programmed to calculate velocity of the dielectric particle due to negative DEP.

8. The system of claim 5, wherein the computer is further programmed to generate a spectrum of velocity as a function of frequency for the dielectric particle.

9. The system of claim 8, wherein the spectrum of velocity as a function of frequency is compared to a standard curve or a second spectrum of velocity as a function of frequency for a second dielectric particle.

10. The system of claim 9, wherein the standard curve or the second spectrum is for a known biomarker of interest.

11. The system of claim 5, wherein the computer is further programmed to calculate cross over frequency.

12. The system of claim 5, wherein the computer is further programmed to calculate the center of mass of the dielectric particle.

13. A method for measuring an effect of a non-uniform electric field on a dielectric particles, comprising:
(a) generating the non-uniform electric field;
(b) changing frequency of the non-uniform electric field;
(c) capturing a series of images of the dielectric particles during exposure to the non-uniform electric field and the changing frequency over a specified time interval; and
(d) detecting changes between the images in the series of images to determine the effect of the non-uniform electric field on the dielectric particles.

14. The method of claim 1, and further comprising providing a start frequency that induces positive DEP effect until the dielectric particle is attracted to the electrode, and then providing another frequency to induce negative DEP.

15. The method of claim 14, and further comprising using the images to calculate velocity of the dielectric particle due to negative DEP.

16. The method of claim 15, and further comprising generating a spectrum of velocity as a function of frequency for the dielectric particle.

17. The method of claim 16, and further comprising contacting the dielectric particle with a sample that possibly contains an analyte of interest, wherein the dielectric particle is functionalized with moieties for binding the analyte of interest.

18. The method of claim 17, and further comprising comparing the spectrum of velocity as a function of frequency for the dielectric particle with a standard curve or spectrum for: (a) a dielectric particle functionalized with moieties for binding the analyte of interest that has not been exposed to a sample, and/or (b) a dielectric particle functionalized with moieties for binding the analyte of interest that has been exposed to a second sample known to contain the analyte of interest.

19. The method of claim 18, wherein the sample is a biological sample obtained from a subject and the analyte of interest is a biomarker for a condition of interest.

20. The method of claim 19, wherein the results of the comparison are used for predicting, diagnosing, providing a prognosis, and/or monitoring treatment for the condition of interest.
